(19) United States
(12) Patent Application Publication
(10) Pub. No.: US 2023/0103651 A1
(43) Pub. Date: Apr. 6, 2023

(54) SYSTEM FOR PROVIDING WOUND DRESSING PORT AND ASSOCIATED WOUND DRESSING

(71) Applicant: Smith & Nephew, Inc., Memphis, TN (US)

(72) Inventors: Julia-Rose Anderson, Melrose, MA (US); Richard M. Braga, North Easton, MA (US); Deirdre McKeown, Boston, MA (US); Adam M. Ruggles, Chicago, IL (US); Chirag B. Shah, North Attleboro, MA (US); David R. Swisher, St. Charles, MO (US); Mark A. Vess, Hanson, MA (US); Ronald F. Vitaris, Worcester, MA (US)

(21) Appl. No.: 17/900,671

(22) Filed: Aug. 31, 2022

Related U.S. Application Data
(63) Continuation of application No. 16/120,056, filed on Aug. 31, 2018, now abandoned.
Application No. 16/120,056 is a continuation-in-part of application No. 15/967,417, filed on Apr. 30, 2018, now abandoned.
Application No. 15/967,417 is a continuation of application No. 14/688,275, filed on Apr. 16, 2015, now Pat. No. 9,956,329.
Application No. 14/688,275 is a continuation of application No. 12/044,051, filed on Mar. 7, 2008, now Pat. No. 9,033,942.
Application No. 16/120,056 is a continuation-in-part of application No. 16/029,369, filed on Jul. 6, 2018, now abandoned.
Application No. 16/029,369 is a continuation of application No. 14/696,211, filed on Apr. 24, 2015, now Pat. No. 10,016,545.
Application No. 14/696,211 is a continuation of application No. 13/218,689, filed on Aug. 26, 2011, now Pat. No. 9,017,302.
Application No. 13/218,689 is a continuation of application No. 12/176,773, filed on Jul. 21, 2008, now Pat. No. 8,021,347.
Application No. 16/120,056 is a continuation-in-part of application No. 15/872,810, filed on Jan. 16, 2018, now Pat. No. 10,828,404.
Application No. 15/872,810 is a continuation of application No. 14/333,026, filed on Jul. 16, 2014, now Pat. No. 9,889,241.
Application No. 14/333,026 is a continuation of application No. 13/571,548, filed on Aug. 10, 2012, now Pat. No. 8,784,392.
Application No. 13/571,548 is a division of application No. 12/475,954, filed on Jun. 1, 2009, now Pat. No. 8,298,200.

(51) Int. Cl. A61F 13/02; A61M 1/00; A61F 13/00; A61M 27/00

(52) U.S. Cl. CPC A61F 13/0226; A61F 13/0206; A61M 1/915; A61F 13/00068; A61M 1/912; A61F 13/0216; A61F 13/025; A61F 13/02; A61F 13/0266; A61M 1/964; A61F 2013/0028; A61M 27/00; A61M 1/74; A61M 2205/7536; A61M 1/86

(57) ABSTRACT

A wound dressing for use in a vacuum wound therapy treatment includes a backing layer for positioning over a wound to define a reservoir in which a reduced pressure may be maintained over the wound. A portal member affixed to the backing layer provides a connection to a reduced pressure source through an opening in an ambient surface. A primary port extends between the opening and a primary aperture in a reservoir surface to providing fluid communication between the reservoir and the reduced pressure source. At least one supplemental port establishes fluid communication between the primary port and a supplemental aperture in the reservoir surface that is distinct and substantially spaced from the primary aperture.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application is a continuation-in-part of co-pending U.S. application Ser. No. 15/967,417, filed Apr. 30, 2018, which is a continuation of U.S. application Ser. No. 14/688,275, filed Apr. 16, 2015 and issued as U.S. Pat. No. 9,956,329, which is a continuation of U.S. application Ser. No. 12/044,051, filed Mar. 7, 2008 and issued as U.S. Pat. No. 9,033,942.

This application is also a continuation-in-part of co-pending U.S. application Ser. No. 16/029,369, filed Jul. 6, 2018, which is a continuation of U.S. application Ser. No. 14/696,211, filed Apr. 24, 2015 and issued as U.S. Pat. No. 10,016,545, which is a continuation of U.S. application Ser. No. 13/218,689, filed Aug. 26, 2011 and issued as U.S. Pat. No. 9,017,302, which is a continuation of U.S. application Ser. No. 12/176,773, filed Jul. 21, 2008 and issued as U.S. Pat. No. 8,021,347.

This application is also a continuation-in-part of co-pending U.S. application Ser. No. 15/872,810, filed Jan. 16, 2018, which is a continuation of U.S. application Ser. No. 14/333,026, filed on Jul. 16, 2014 and issued as U.S. Pat. No. 9,889,241, which is a continuation of U.S. application Ser. No. 13/571,548, filed on Aug. 10, 2012 and issued as U.S. Pat. No. 8,784,392, which is a divisional of U.S. application Ser. No. 12/475,954, filed on Jun. 1, 2009 and issued as U.S. Pat. No. 8,298,200.

The disclosures of all these prior applications are hereby incorporated herein by reference in their entireties and are to be to be considered a part of this specification.

### BACKGROUND

### Technical Field

In some embodiments, the present disclosure relates generally to a wound dressing for treating an open wound with a vacuum wound therapy treatment. In particular, the disclosure relates to a dressing having a portal member providing a connection to a vacuum system such that a reservoir over the wound may be evacuated.

In some embodiments, the present disclosure relates generally wound dressings, and in particular to a delivery apparatus for the application of thin film dressings over a wound for use in a treatment such as negative wound pressure therapy.

In some embodiments, the present disclosure relates generally to treating a wound with negative or reduced pressure. In particular, the disclosure relates to a system for providing continual drainage of fluids from a wound site to a collection canister.

### Background of Related Art

The body's natural wound healing process is a complex series of events beginning at the moment of injury. Initially the body reacts by delivering proteins and other factors to the wound through the blood stream to minimize the damage. Blood clots to prevent blood loss while cells engulf bacteria and debris to carry it away from the wound site. Next, the body begins to repair itself in a stage of healing often referred to as the proliferative phase. This phase is characterized by the deposition granulation tissue in the wound bed. Granulation tissue provides a base structure over which cells may migrate inwardly from the periphery to close the wound. Finally the process ends as collagen gives strength to new tissue over time often forming a scar.

One technique for promoting the natural healing process, particularly, but not exclusively during the proliferative phase, is known as vacuum wound therapy (V WT). Application of a reduced pressure, e.g. sub-atmospheric, to a localized reservoir over a wound has been found to assist in closing the wound. The reduced pressure may be effective to promote blood flow to the area to stimulate the formation of granulation tissue and the migration of healthy tissue over the wound by the natural process. Also a reduced pressure may assist in removing fluids exuding from the wound, which may inhibit bacterial growth. This technique has proven effective for chronic or non-healing wounds, but has also been used in for other purposes such as postoperative wound care.

The general VWT protocol provides for the introduction of an absorbent filler material, such as non-reticulated foams, non-woven fabrics, or gauze, into the wound to absorb exudates. The wound and the absorbent filler material may then be overlaid by a flexible cover layer such as a polymeric film, for example, to establish a vacuum reservoir over the wound where a reduced pressure may be applied by individual or cyclic evacuation procedures. To allow the reduced pressure to be maintained over time, the cover layer may include an adhesive periphery that forms a substantially fluid tight seal with the healthy skin surrounding the wound.

Although some procedures may employ a micro-pump contained within the vacuum reservoir, most V WT treatments apply a reduced pressure using an external vacuum source. Thus, fluid communication between the vacuum source and the reservoir must be established. To this end, a cover layer will often include a connector or portal member to which air hoses from an external vacuum system may be connected.

An aspect of concern in a VWT treatment is the migration of the filler material and wound exudates in the direction of airflow when the reduced pressure is applied. These substances may be deposited on surfaces within a portal member, for example, and over time cause the obstruction or complete occlusion of these portal members. This phenomenon may limit the level of exudate flow from the wound area, or may even prohibit the application of a reduced pressure to the wound area, thus limiting or defeating the effectiveness of the VWT treatment. Accordingly, a need exists for a dressing suitable for use in a VWT procedure.

Wound dressings are generally placed over a wound to protect and promote healing of the wound. One type of wound dressing consists essentially of a thin membrane of a polymer or similar material, coated on an underside with a pressure-sensitive adhesive. The adhesive may adhere the dressing to healthy skin surrounding the wound such that the dressing provides an effective bacterial barrier to protect the wound from contamination. Because of their extremely elastic nature, thin polymeric film dressings may readily conform to irregular contours of a patient's skin while promoting patient movement and comfort. This type of dressing may also be sufficiently transparent to permit visual inspection of the wound without the need for removing the dressing and exposing the wound to contaminants in the environment.

One technique that may utilize a thin film dressing may be described as negative wound pressure therapy (NWPT). The thin film dressing may be positioned to form a substantially fluid tight seal with the skin surrounding the wound to define a reservoir over the wound where a negative pressure may be maintained. The reservoir subjects the wound to a sub-atmospheric pressure to effectively draw wound fluid, including liquid exudates, from the wound with, e.g., a vacuum pump. Vacuum pressure may be applied continuously, or in varying intervals, depending on the nature and severity of the wound. This technique has been found to promote blood flow to the wound area, stimulate the formation of granulation tissue and encourage the migration of healthy tissue over the wound. This type of treatment may subject a thin film dressing 10 repeated changes of size and shape, taking advantage of the flexibility of the dressing.

The flexibility of a thin film dressing may, however, present difficulties in the application of the dressing to a wound site. For example, the dressing may tend to fold, wrinkle and adhere to itself. To mitigate these tendencies, a delivery layer may be supplied with the dressing to temporarily support the dressing until the dressing is applied. When a thin film dressing is applied as part of an NWPT treatment, additional concerns arise including properly sizing the dressing and appropriately locating a vacuum port relative to the wound. Accordingly, a need exists for a composite dressing and delivery apparatus suitable for use in conjunction with an NWPT treatment.

Various techniques to promote healing of a wound involve providing suction to the wound. For example, a vacuum source may serve to carry wound exudates away from the wound, which may otherwise harbor bacteria that inhibit the body's natural healing process. One particular technique for promoting the body's natural healing process may be described as negative pressure wound therapy (NPWT). This technique involves the application of a reduced pressure, e.g. sub-atmospheric, to a localized reservoir over a wound. Sub-atmospheric pressure has been found to assist in closing the wound by promoting blood flow to the area, thereby stimulating the formation of granulation tissue and the migration of healthy tissue over the wound. This technique has proven effective for chronic or non-healing wounds, but has also been used for other purposes such as post-operative wound care.

The general NPWT protocol provides for covering the wound with a flexible cover layer such as a polymeric film, for example, to establish a vacuum reservoir over the wound where a reduced pressure may be applied by individual or cyclic evacuation procedures. To allow the reduced pressure to be maintained over time, the cover layer may include an adhesive periphery that forms a substantially fluid tight seal with the healthy skin surrounding the wound.

Although some procedures may employ a micro-pump contained within the vacuum reservoir, most NPWT treatments apply a reduced pressure using an external vacuum source. Fluid communication must therefore be established between the reservoir and the vacuum source. To this end, a fluid port is coupled to the cover layer to provide an interface for an exudate conduit extending from the external vacuum source. Fluid being drained from the reservoir through the exudate conduit tends to stagnate with slow fluid buildup. This stagnation results in interrupted and/or incomplete fluid drainage. Accordingly, it would be beneficial to have a negative pressure wound therapy system that included a controlled or fixed “leak” to provide for continuous and/or complete fluid drainage.

### SUMMARY

In some embodiments, the present disclosure describes a wound dressing for use in a vacuum wound therapy treatment. The wound dressing includes a backing layer for positioning over a wound to define a reservoir in which a reduced pressure may be maintained over the wound. A portal member is affixed to the backing layer such that an ambient surface of the portal member may be fluidly isolated from a reservoir surface of the portal member. The backing layer defines a boundary between the ambient surface and the reservoir surface when the backing layer is positioned over a wound. The portal member comprises a primary port extending between an opening in the ambient surface and a primary aperture in the reservoir surface. The opening is adapted for connection to a suction device and the primary aperture is adapted for establishing fluid communication with the reservoir. The portal member further comprises at least one supplemental port establishing fluid communication between the primary port and a supplemental aperture in the reservoir surface. The supplemental aperture is distinct and substantially spaced from the primary aperture.

The portal member may include a plurality of supplemental ports extending radially outwardly from the primary port. The primary and supplemental apertures may be distributed substantially over the entire reservoir surface. The may be molded from a flexible polymeric or elastomeric material and may include an attachment flange adapted for connection of an adhesively coated film.

The backing layer of the wound dressing may comprise a flexible polymeric membrane. The backing layer may include an adhesive coating adapted to affix the backing layer over the wound and to provide a substantially fluid tight seal around the perimeter of the wound. The adhesive coating may be interrupted such that an intermediate portion of the backing layer remains uncoated.

In some embodiments, a vacuum wound therapy system includes a contact layer and an absorbent filler positioned in a wound bed, and a portal member affixed to a backing layer positioned over the wound bed to define a vacuum reservoir. The portal member includes a primary port extending between an opening in an ambient surface and a primary aperture in a reservoir surface, and at least one supplemental port extending between the primary port and a supplemental aperture distinct and substantially spaced from the primary aperture.

The contact layer may be formed from a conical apertured film to promote unidirectional flow of exudates from the wound. The absorbent filler material may include a single strand of a polyolefin filament. Also, a vacuum system in fluid communication with the vacuum reservoir may include a vacuum source, a collection canister and a one-way valve.

In some embodiments, the present disclosure relates to a composite wound dressing and delivery apparatus includes a substantially transparent dressing layer having a lower surface that is coaled with a pressure sensitive for applying the dressing layer over a wound to define a reservoir in which a negative pressure may be maintained. A substantially transparent backing layer adhered to the lower surface of the dressing layer in a releasable manner, and a vacuum port is centrally located on the dressing layer. The vacuum port is adapted to provide fluid communication between a vacuum source and the reservoir through the dressing layer. A targeting grid associated with either the dressing layer or the backing layer includes regularly spaced reference marks along at least two axes extending from the vacuum port.

The targeting grid may be applied to the backing layer, and may include rule marks associated with two orthogonal axes extending from the vacuum port such that the targeting grid is arranged for Cartesian measurement of a distance to the vacuum port. At least a portion of the rule marks may be associated with numerical markers corresponding to units of a standard measurement system, and the numerical markers may identify a number of the units that is twice a distance from a center of the vacuum port.

The targeting grid may include orthogonal gridlines, including major gridlines and minor gridlines where the major gridlines are adapted to appear more prominent than the minor gridlines. The targeting grid may alternatively include curvilinear rule lines arranged around the vacuum port such the targeting grid is arranged for radial measurement of a distance to the vacuum port.

The apparatus may also include a substantially transparent delivery layer adhered to the upper surface of the dressing layer in a releasable manner. The backing layer may include a first identifier prominently visible thereon and the delivery layer may include a second identifier obscured by the backing layer such that the second identifier is revealed by the removal of the backing layer. The first and second identifiers may thus indicate an order in which the backing layer and delivery layer should be removed from the dressing layer.

The delivery layer may include a slit extending between a central opening and an exterior edge of the delivery layer to permit the delivery layer to be removed from the dressing layer when a vacuum tube is coupled to the vacuum port. The delivery layer may comprise a pair of opposed tabs protruding beyond the extents of the dressing layer. The apparatus also include a dressing layer that is generally triangular in shape.

In some embodiments, a composite wound dressing and delivery apparatus includes a substantially transparent dressing layer having a lower surface and an upper surface. The lower surface is coaled with a pressure sensitive adhesive such that the dressing layer may form a fluid tight seal over a wound to define a reservoir in which a negative pressure may be maintained. The apparatus also includes substantially transparent backing layer adhered to the lower surface of the dressing layer in a releasable manner, and a substantially transparent delivery layer adhered to the upper surface of the dressing layer in a releasable manner. A vacuum port is centrally located on the dressing layer, and is adapted to provide fluid communication between a vacuum source and the reservoir through the dressing layer. A targeting grid is associated with the dressing layer, backing layer or the delivery layer, and includes regularly spaced reference marks along at least two axes extending from the vacuum port. Numerical markers correspond to units of a standard measurement system, and identify a number of the units that is twice the distance from a center of the vacuum port.

In some embodiments, a negative wound pressure therapy kit includes a composite wound dressing, a delivery apparatus and a patch. The composite apparatus includes a dressing layer configured for placement over a wound to define a reservoir over the wound in which a negative pressure may be maintained, a backing layer adhered to the dressing layer in a releasable manner and a vacuum port for providing fluid communication through the dressing layer. The vacuum port exhibits a predetermined geometry. The patch includes a patch layer and a backing layer. The patch layer has an opening therein that exhibits a geometry substantially similar to the geometry of the vacuum port.

The kit may further include a filler material adapted for placement within a wound to capture wound exudates, a wound contact layer adapted for placement adjacent the wound to promote unidirectional flow of wound exudates, or a canister adapted for placement exterior to the wound for the collection of wound exudates.

In some embodiments, a system for subatmospheric pressure therapy in connection with healing a wound is provided. The system includes a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system further includes an exudate conduit in fluid communication with the wound dressing and the collection canister for collecting exudate remove from the reservoir and deposited in the collection canister under influence of the vacuum source and a vent conduit in fluid communication with the collection canister and the wound dressing for introducing air into the reservoir to facilitate flow of exudate through the exudate conduit.

The vent conduit may define an internal dimension less than a corresponding internal dimension of the exudate conduit. The exudate conduit and the vent conduit may include independent tube segments, or instead may include integral tube segments. A filter may be in fluid communication with the vent conduit. The filter includes a hydrophobic material. The filter may instead or additionally include a bacterial filter.

In some embodiments, the present disclosure relates to a system for subatmospheric pressure therapy in connection with healing a wound including a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system further includes an exudate conduit in fluid communication with the wound dressing and the collection canister for collecting exudate removed from the reservoir and deposited in the collection canister under influence of the vacuum source and a vent mounted to the wound dressing, the vent being selectively movable between a closed position and an open position, the vent permitting ingress of air within the reservoir when in the open position.

The vent may include a flap mounted to the wound dressing cover, the flap being movable between the closed position and the open position. The flap may be releasably securable in the closed position with an adhesive. A filter membrane may be mounted adjacent the flap. The filter membrane may include a hydrophobic material. The filter membrane may instead or additionally include a bacterial filter.

In some embodiments, the present disclosure relates to a system for subatmospheric pressure therapy in connection with healing a wound including a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system further includes an exudate conduit in fluid communication with the wound dressing and the collection canister for collecting exudate removed from the reservoir and deposited in the collection canister under influence of the vacuum source and a filtered air vent mounted to the wound dressing cover, the filtered air vent adapted to permit ingress of air within the reservoir to facilitate flow of exudate through the exudate conduit.

In some embodiments, the present disclosure relates to a system for subatmospheric pressure therapy in connection with healing a wound including a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system also includes a wound port operatively connected to the wound dressing in fluid communication with the reservoir. The wound port includes a vacuum port and at least one tube piercing through the wound port into the reservoir, the tube being operable to allow ambient air into the reservoir. The system further includes an exudate conduit in fluid communication with the wound port and the collection canister for collecting exudate removed from the reservoir and deposited in the collection canister under influence of the vacuum.

In some embodiments, the present disclosure relates to a system for subatmospheric pressure therapy in connection with healing a wound including a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system also includes a wound port operatively connected to the wound dressing in fluid communication with the reservoir. The wound port includes a vacuum port and a plurality of holes arranged circumferentially around the wound port, the plurality of holes being operable to allow ambient air into the reservoir. The system further includes an exudate conduit in fluid communication with the wound port and the collection canister for collecting exudate removed from the reservoir and deposited in the collection canister under influence of the vacuum.

In some embodiments, the present disclosure relates to a system for subatmospheric pressure therapy in connection with healing a wound including a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system also includes a wound port operatively connected to the wound dressing in fluid communication with the reservoir. The wound port includes a vacuum port and an orifice being operable to allow ambient air into the reservoir. The system further includes an exudate conduit in fluid communication with the wound port and the collection canister for collecting exudate removed from the reservoir and deposited in the collection canister under influence of the vacuum.

In some embodiments, the present disclosure relates to a system for subatmospheric pressure therapy in connection with healing a wound including a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system also includes a wound port operatively connected to the wound dressing in fluid communication with the reservoir. The system further includes an exudate conduit in fluid communication with the wound port and the collection canister for collecting exudate removed from the reservoir and deposited in the collection canister under influence of the vacuum. The exudate conduit has a first conduit for providing a pathway for the exudate between the reservoir and the collection canister and a second conduit in fluid communication with ambient atmosphere and the wound dressing for introducing air into the reservoir to facilitate flow of exudate through the exudate conduit.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is an exploded perspective view of a vacuum wound therapy system in accordance with the present disclosure;

FIG. 2 is an orthographic view of a wound facing side of the portal member of FIG. 1;

FIG. 3 is a cross sectional view taken along the line 3-3 of FIG. 2;

FIG. 4 is a cross sectional view of the vacuum wound therapy system of FIG. 1 installed over wound on a patient; and

FIG. 5 is a view similar to FIG. 4 depicting an alternate embodiment of a vacuum wound therapy system.

FIG. 6 is a cross sectional view of a thin film wound dressing as applied in an NWPT treatment apparatus;

FIG. 7 is an exploded perspective view of a composite dressing and delivery apparatus for the thin film wound dressing of FIG. 6 including a dressing layer, a backing layer and a delivery layer;

FIG. 8 is a flow diagram describing a process for changing the dressing of FIG. 6;

FIGS. 9A through 11B are top plan views of alternate embodiments of a composite dressing and delivery apparatus (designated “A”), and corresponding views of the dressing layers prepared for application with backing layers removed (designated “B”);

FIGS. 12A through 14B are top plan views of patches for use with a thin film dressing as supplied (designated “A”), and corresponding views of the patches prepared for application with backing layers removed (designated “B”):

FIG. 15A is a perspective view of a thin film dressing as applied in an NWPT treatment;

FIGS. 15B and 15C are top plan views of the thin film dressing of FIG. 15A as supplied (designated “B”) and as prepared for application (designated “C”);

FIG. 16A is a perspective view of a thin film dressing as applied in an NWPT treatment;

FIGS. 16B and 16C are top plan views of the thin film dressing of FIG. 16A as supplied (designated “B”) and as prepared for application (designated “C”);

FIG. 17A is a perspective view of a thin film dressing as applied in an NWPT treatment; and

FIGS. 17B and 17C are top plan views of the thin film dressing of FIG. 16A as supplied (designated “B”) and as prepared for application (designated “C”).

FIG. 18 depicts an embodiment of a NPWT system in accordance with the present disclosure;

FIG. 19 depicts an embodiment of an NPWT treatment apparatus including a vent conduit;

FIG. 20A is a partial cross sectional view of the conduits of the NPWT treatment apparatus of FIGS. 18 and 19 connected in an alternate configuration;

FIG. 20B is a partial cross sectional view of an alternative embodiment of the fluid of FIGS. 18 and 19;

FIG. 21 is a cross sectional view of an alternative embodiment of the wound dressing in accordance with the present disclosure;

FIGS. 22A and 22B depict alternative embodiments of the wound dressing in accordance with the present disclosure;

FIGS. 23A and 23B depict alternative embodiments of the wound dressing in accordance with the present disclosure;

FIGS. 24A and 24B depict alternative embodiments of the wound dressing in accordance with the present disclosure;

FIGS. 25A and 25B depict alternative embodiments of the wound dressing in accordance with the present disclosure;

FIG. 26 depicts an alternative embodiment of the wound port in accordance with the present disclosure;

FIGS. 27A and 27B depict alternative embodiments of the wound port in accordance with the present disclosure; and

FIGS. 28A-28D depict alternative embodiments of the wound port in accordance with the present disclosure.

### DETAILED DESCRIPTION OF THE PREFERRED EMBODIMENT

Various embodiments of the present disclosure provide negative pressure wound therapy systems (or apparatus) including a collection canister having a chamber to collect wound fluids. Embodiments of the presently disclosed negative pressure wound therapy systems are generally suitable for use in applying negative pressure to a wound to facilitate healing of the wound in accordance with various treatment modalities. Embodiments of the presently disclosed negative pressure wound therapy systems are entirely portable and may be worn or carried by the user such that the user may be completely ambulatory during the therapy period. Embodiments of the presently disclosed negative pressure wound therapy apparatus and components thereof may be entirely reusable or may be entirely disposable after a predetermined period of use or may be individually disposable whereby some of the components are reused for a subsequent therapy application.

Hereinafter, embodiments of the presently disclosed negative pressure wound therapy systems and embodiments of the presently disclosed sensors for use in negative pressure wound therapy systems will be described with reference to the accompanying drawings. Like reference numerals may refer to similar or identical elements throughout the description of the figures. As used herein, “wound exudate”, or, simply, “exudate”, generally refers to any fluid output from a wound, e.g., blood, serum, and/or pus, etc. As used herein, “fluid” generally refers to a liquid, a gas or both.

### Wound Dressing Port and Associated Wound Dressing

A wound dressing for use in a vacuum wound therapy treatment includes a backing layer for positioning over a wound to define a reservoir in which a reduced pressure may be maintained over the wound. A portal member affixed to the backing layer provides a connection to a reduced pressure source through an opening in an ambient surface. A primary port extends between the opening and a primary aperture in a reservoir surface to providing fluid communication between the reservoir and the reduced pressure source. At least one supplemental port establishes fluid communication between the primary port and a supplemental aperture in the reservoir surface that is distinct and substantially spaced from the primary aperture.

The wound dressing of the present disclosure promotes healing of a wound by providing a reservoir over the wound where a reduced pressure may be maintained. The reservoir subjects the wound to a sub-atmospheric pressure to effectively draw wound fluid, including liquid exudates, from the wound without the continuous use of a vacuum pump. Hence, vacuum pressure may be applied once, or in varying intervals depending on the nature and severity of the wound. The use of a wound dressing in this manner has been found to promote healing by reducing the probability of infection, stimulating the deposition of granulation tissue and other beneficial processes, The wound dressing of the present disclosure includes a backing layer affixed to a portal member for enhancing the effect of a vacuum wound therapy treatment.

The attached figures illustrate exemplary embodiments of the present disclosure and are referenced to describe the embodiments depicted therein. Hereinafter, the disclosure will be described in detail by explaining the figures wherein like reference numerals represent like parts throughout the several views.

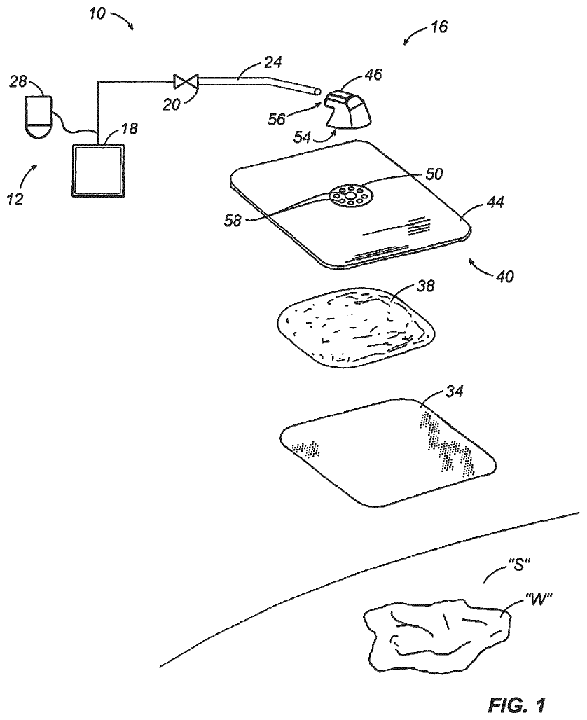

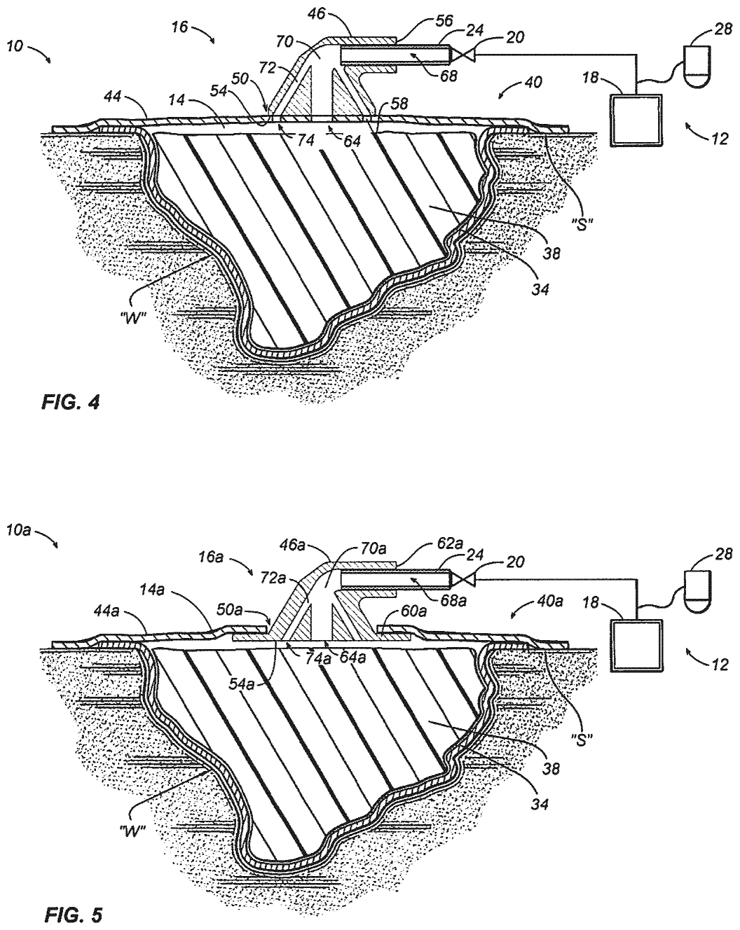

Referring initially to FIG. 1, a vacuum wound therapy system according to the present disclosure is depicted generally as 10 for use on a wound “w” surrounded by healthy skin “s.” The vacuum wound therapy system 10 includes a vacuum system 12 in fluid communication with a vacuum reservoir 14 (FIG. 4) defined by or within wound dressing 16. The vacuum system 12 includes a vacuum source 18 coupled to the dressing 16 through a one-way valve 20 and a suction device such as vacuum tube 24. A collection canister 28 may be provided for wound drainage and debris. The vacuum system 12 is adapted to provide a reduced pressure to the vacuum reservoir 14 appropriate to stimulate healing of the wound “w.” A more detailed description of an appropriate vacuum system 12 is found in commonly assigned U.S. Patent Application Publication 2007/0066946, the entire contents of which are incorporated herein by reference.

Wound dressing 16 generally includes a contact layer 34, filler 38 and a cover layer 40. Cover layer 40 includes a backing layer 44 and a portal member 46. Portal member 46 is configured to establish fluid communication between vacuum reservoir 14 and vacuum system 12 by providing an interface for a suction device such as vacuum tube 24. Each layer of wound dressing 16 is described in greater detail below.

Contact layer 34 may be sufficiently conformable to be positioned in direct contact with an irregularly shaped surface of a wound bed “w.” A thin film of polyethylene or other suitable non-adherent material may form the contact layer 34 to limit the adherence of filler 38 and other substances to the wound “w.” Apertures or perforations in the film permit fluids to pass through the contact layer 34, allowing for the sub-atmospheric pressure to penetrate into the wound “w” and for exudates to flow freely out of the wound “w.” By selecting an appropriate film material, the passage of wound exudate through contact layer 34 may be controlled so as to be substantially unidirectional to prevent wound exudate from flowing back into the wound. To promote a unidirectional flow, a conical apertured film, such as VENTEX™ by Kendall Corp., a division of Covidien, or apertured films by Tredegar Film Products of Richmond, Va., may be selected for forming contact layer 34. Unidirectional flow of exudates may also be promoted by the selection of other materials including a lamination of layers having varying absorptive characteristics. One exemplary material, which may be used as a contact layer is sold under the trademark XEROFLO® by Kendall Corp., a division of Covidien.

Filler 38 may be arranged over contact layer 34 to fill wound “w” to the level of the surrounding healthy skin “s.” An absorbent material such as non-woven gauze or reticulated foam may be used for filler 38 to trap any exudate that migrates through contact layer 34. An antimicrobial dressing sold under the trademark KERLIX® by Kendall Corp., a division of Covidien, may be suitable for use as filler 38. To prevent adhesion to the wound “w,” the filler 38 may also comprise a material configured such that any stray fibers do not tend to protrude through apertures of contact layer 34 where they may become engulfed by newly forming granulation tissue. One particular type of material exhibiting this characteristic is often referred to as “tow.” The manufacturing process for synthetic fibers often includes an extrusion of an indeterminate length of continuous filaments, which are spun together to form fibers. It is the continuous lengths of un-spun filaments which are referred to as tow. A single length of tow formed from a hydrophobic material such as polyolefin may be laid in the wound bed “w” to form filler 38. This arrangement allows for a complete removal of filler 38 when the dressing 16 is changed without re-injuring the wound “w.”

Cover layer 40 may be supplied as a single unit including both backing layer 44 and portal member 46, or alternatively, these items may be provided individually and assembled at the time of installation over the wound “w.” Cover layer 40 may be placed over the wound “w” enclosing the contact layer 34 and filler 38 therein.

Backing layer 44 may be formed from a flexible polymeric membrane or film to serve as a fluid barrier to allow for a sub-atmospheric pressure to be established in vacuum reservoir 14, and also as a microbial barrier preventing contaminants from entering the wound area. For example, backing layer 44 may comprise a polyurethane film having a thickness from about 0.8 mils to about 1.0 mils. Preferably, the backing layer 44 is formed from a moisture vapor permeable membrane to promote the exchange of oxygen and moisture vapor between the wound site and the atmosphere. One exemplary material is a transparent membrane sold under the trade name POLYSKIN® II by Kendall Corp., a division of Covidien. Other materials which may be suitable for use in a backing layer include the thin films marketed under the names TEGADERM™ by 3M of St. Paul, Minn. and OPSITE™ by Smith and Nephew PLC of London, UK.

The backing layer 44 of cover layer 40 is adapted to extend laterally beyond the perimeter of the wound bed “w” so as to contact the healthy skin “s” to form a seal thereto. To form the seal, backing layer 44 may be equipped with an adhesive coating on all, or any appropriate portion of a wound facing side. The adhesive coating may comprise, for example, a medical-grade, pressure-sensitive adhesive adapted to provide a fluid-tight and bacteria-tight seal around the wound “w.” Thus exudate cannot escape through the edges of the dressing 16 and external air and contaminants may not enter the wound area. To provide such a seal, the adhesive coating may, for example, be in the range from about 1 mil to about 10 mils thick. In general, a high peal-strength adhesive may be used to resist inadvertent lift-off, roll or “flagging,” i.e., a failure of the dressing to adhere to itself, at the edges of the cover layer 40. The adhesive may include, for example, those adhesives included with the dressing sold under the trademark ULTEC® Hydrocolloid by Kendall Corp., a division of Covidien.

Backing layer 44 includes a connective region 50 to permit of connection of portal member 46. Connective region 50 receives a reservoir surface 54 of portal member 46 to fluidly isolate the reservoir surface 54 from an ambient surface 56 on the portal member 46 except through portal member 46. As illustrated in FIGS. 1 and 4, connective region 50 may be adapted to receive reservoir surface 54 directly such that an adhesive coating on either reservoir surface 54 or connective region 50 affixes the portal member 46 to the backing layer 44. Backing layer 44 defines a boundary between reservoir surface 54 and ambient surface 56. This arrangement permits isolation of reservoir surface 54 from reservoir 14, i.e. reservoir surface 54 may be disposed outside of reservoir 14. Perforations 58 may be formed through the connective region 50 to permit fluids to be drawn from reservoir 14. Alternatively, as illustrated in FIG. 5, backing layer 44a may include an entry 50a dimensioned such that a portion of portal member 46a protrudes through backing layer 44a. An appropriately positioned adhesive coating may form a seal between a wound facing side of backing layer 44a and an oppositely oriented surface of a flange 60a. This arrangement permits reservoir surface 54a to be positioned within reservoir 14a fluidly isolating reservoir surface 54a from ambient surface 56a. Other arrangements are contemplated for affixing a portal member 46, 46a to a backing layer 44, 44a, which may include other parts such as sealing rings (not shown).

Backing layer 44 may include an interrupted adhesive coating such that only the portions of backing layer 44 forming a seal are coated. For example, backing layer 44 may include a band of adhesive coating around the periphery to form a seal with the skin “s,” and another band near the connective region 50 to form a seal with the portal member 46. An intermediate portion of backing layer 44 may remain uncoated to limit the adherence of backing layer 44 to the filler 38, contact layer 34 or other surfaces. Such an arrangement may facilitate removal of the dressing 16 without re-injuring the wound “w.”

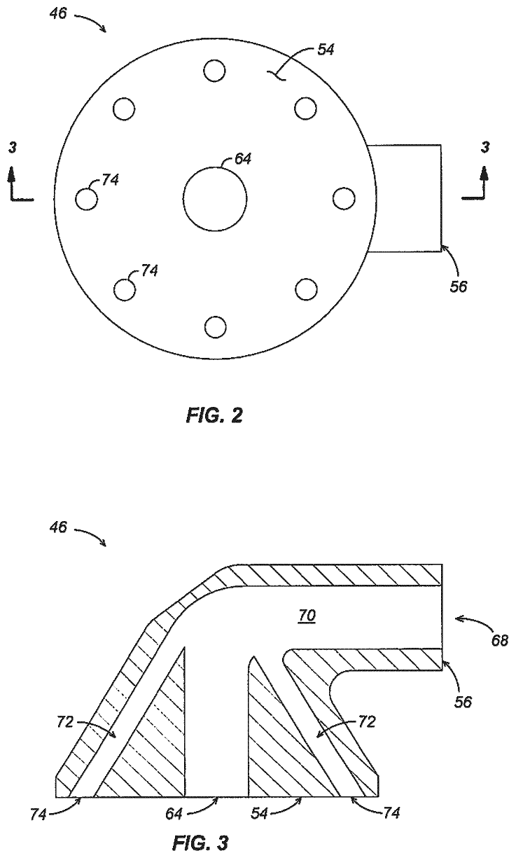

Referring now to FIGS. 2 and 3, a portal member 46 may be formed as a rigid component, or alternatively portal member 46 may be molded from a flexible polymeric or elastomeric material. Flexibility in portal member 46 facilitates the placement of wound dressing 16 on curved areas of a patient's skin “s.” Portal member 46 includes a reservoir surface 54 and an ambient surface 56. The reservoir surface 54 is adapted to be positioned such that a primary aperture 64 therein may fluidly communicate with vacuum reservoir 14, through a perforation 58 for example. Ambient surface 56 may be positioned in the environment surrounding dressing 16 and includes an opening 68 adapted for connection to a suction device such as vacuum tube 24 of vacuum system 12. A primary port 70 is defined between the primary aperture 64 and the opening 68 to permit fluid communication between the reservoir 14 and the vacuum system 12.

Branching from primary port 70 is a radial array of supplemental ports 72 extending to reservoir surface 54. Each of the supplemental ports 72 terminates at a supplemental aperture 74 distinct from and substantially spaced from primary aperture 64. Any number, distribution and arrangement of supplemental pons 72 may be included to distribute supplemental openings 74 over the reservoir surface 54. As FIG. 2 illustrates supplemental apertures 74 may be positioned near a periphery of reservoir surface 54 such that fluids may be drawn from reservoir 14 over a collection area spanning the entire area covered by the portal member 46. Any number, size and arrangement of primary and supplemental apertures 64, 74 is contemplated to effectively draw fluids for a particular application.

Referring now to FIG. 4, wound dressing 16 may be installed on a patient to cover a wound “w.” Reservoir 14 is defined by or within wound dressing 16 when applied to the skin “s.” Contact layer 34 is positioned in direct contact with the wound “w” and filler 38 is positioned up to the level of healthy skin “s.” Portal member 46 may be positioned over connective region 50 of backing layer 44 such that the primary and supplemental apertures 64, 74 are aligned with perforations 58. This will allow fluids to be drawn into primary and supplemental ports 70, 72 from reservoir 14. Ambient surface 56 is fluidly isolated from reservoir 14 except through primary and supplemental ports 70, 72. Opening 68 in the ambient surface 56 receives vacuum tube 24, thus fluidly connecting vacuum system 12 with reservoir 14.

Evacuating the reservoir 14 may impart a tendency for filler material 38 and wound exudate to migrate in the direction of airflow. This tendency for migration may result in the obstruction or occlusion of one or more of the primary and supplemental ports 70, 72. However, because portal member 46 may include many apertures 64, 74 distributed over the entire area covered by the portal member 46, the effectiveness of the VWT procedure may persist. If one of the apertures 64, 74 becomes clogged, fluids will be drawn to another one of the apertures 64, 74 that is distinct and substantially spaced from the clogged aperture 64, 74.

Referring now to FIG. 5, an alternate embodiment of a vacuum wound therapy system is depicted generally as 10a. Wound dressing 16a includes a portal member 46a protruding through entry 50a of cover layer 40a. Backing layer 44a adheres to the flange 60a of the portal member 46a and also to the skin “s” such that the reservoir surface 54a and ambient surface 56a are fluidly isolated except through the primary and supplemental ports 70a, 72a. Ambient surface 56a has an opening 68a therein for connection of a suction device such as vacuum tube 24. Fluid communication is thus established between reservoir 14a and vacuum system 12 through primary port 70a, and primary opening 64a. Supplemental ports 72a establish fluid communication between primary port 70a and supplemental apertures 74a in reservoir surface 54a. Supplemental apertures 74a are distinct and substantially spaced from primary aperture 64a. In this arrangement, reservoir surface 54a is enclosed by reservoir 14a.

Portal member 46a includes a radial array of supplemental ports 72a such that apertures 64a, 74a may be distributed substantially over the entire reservoir surface 54a. This arrangement provides a means of keeping a collection area of the port 46a exposed to as much of the dressing 16a as possible.

### Thin Film Wound Dressing

A composite wound dressing and delivery apparatus includes a substantially transparent dressing layer having a lower surface that is coated with a pressure sensitive for applying the dressing layer over a wound to define a reservoir in which a negative pressure may be maintained. A substantially transparent backing layer adhered to the lower surface of the dressing layer in a releasable manner, and a vacuum port is centrally located on the dressing layer. The vacuum port is adapted to provide fluid communication between a vacuum source and the reservoir through the dressing layer. A targeting grid associated with either the dressing layer or the backing layer includes regularly spaced reference marks along at least two axes extending from the vacuum port.

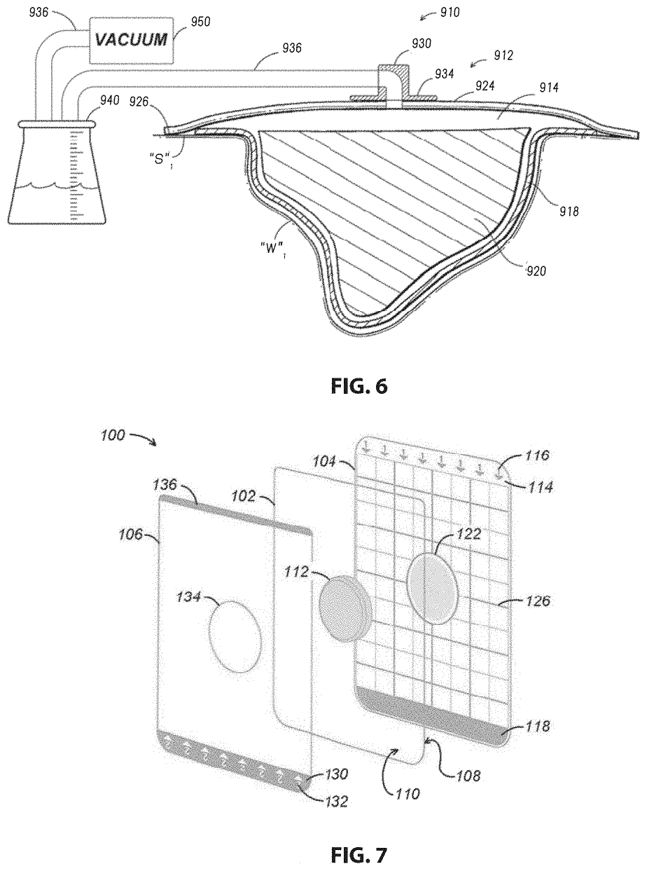

Referring initially to FIG. 6, a conventional NWPT apparatus is depicted generally as 910 for use on a wound “w₁” surrounded by healthy skin “s₁.” The NWPT apparatus 910 includes a wound dressing 912 positioned relative to the wound “w₁” to define a reservoir 914 in which a negative pressure appropriate to stimulate healing may be maintained.

Wound dressing 912 includes a contact layer 918 positioned in direct contact with the bed of wound “w₁” and may be formed from perforated film material. An appropriate perforated material permits the negative pressure applied to the reservoir to penetrate into the wound “w₁,” and also permits exudates to be drawn through the contact layer 918. Passage of wound fluid through the contact layer 918 is preferably unidirectional such that exudates do not flow back into the wound bed. Unidirectional flow may be encouraged by conical or directional apertures formed in the contact layer 918, or a lamination of materials having absorption properties differing from those of contact layer 918. A non-adherent material may be selected such that contact layer 918 does not tend to cling to the wound “w₁” or surrounding tissue when it is removed. One exemplary material that may be used as a contact layer 918 is sold under the trademark XEROFORM® and VENTEX® by Tyco Healthcare Group LP (d/b/a Covidien).

Wound filler 920 is positioned in the wound “w₁” over the contact layer 918 and is intended to allow wound dressing 912 to absorb, capture and/or wick wound exudates. Wound filler 920 is cut to a shape that is conformable to the shape of wound “w₁,” and may be packed up to the level of healthy skin “s₁,” or alternatively, wound filler 920 may overfill the wound “w₁.” An absorbent material such as non-woven gauze, reticulated foam, or alginate fibers may be used for filler 920 to transfer any exudate that migrates through contact layer 918 away from the wound “w₁”. An antimicrobial dressing sold under the trademark KERLIX®AMD by Tyco Healthcare Group LP (d/b/a Covidien), may be suitable for use as filler 920.

Wound dressing 912 also includes a cover layer 924. Cover layer 924 may be positioned over the wound “w₁” to form a substantially fluid-tight seal with the surrounding skin “s₁.” Thus, cover layer 924 may act as both a microbial barrier to prevent contaminants from entering the wound “w₁,” and also a fluid barrier maintaining the integrity of vacuum reservoir 914. Cover layer 924 is preferably formed from a moisture vapor permeable membrane to promote the exchange of oxygen and moisture between the wound “w₁” and the atmosphere, and is preferably transparent permit a visual assessment of wound conditions without requiring removal of the cover layer 924. A membrane that provides a sufficient moisture vapor transmission rate (MVTR) is a transparent membrane sold under the trade name POLYSKIN®II by Tyco Healthcare Group LP (d/b/a Covidien) Cover layer 924 may be customized from a composite dressing and delivery apparatus 100 (FIG. 7) as described in greater detail below.

A vacuum port 930 having a flange 934 may also be included in wound dressing 912 to facilitate connection of the wound dressing 912 to fluid conduit 936. The vacuum port 930 may be configured as a rigid or flexible, low-profile component, and may be adapted to receive a fluid conduit 936 in a releasable and fluid-tight manner. An adhesive on the underside of flange 934 may provide a mechanism for affixing the vacuum port 930 to the dressing 912, or alternatively the flange 934 may be positioned within reservoir 914 (not shown) such that an adhesive on an upper side of the flange 934 affixes the vacuum port 930. However it is affixed to the dressing, a hollow interior of the vacuum port 930 provides fluid communication between the fluid conduit 936 and the reservoir 914. Vacuum port 930 may assume various other forms discussed below.

Fluid conduit 936 extends from the vacuum port 930 to provide fluid communication between the reservoir 914 and collection canister 940. Any suitable conduit may be used for fluid conduit 936 including those fabricated from flexible elastomeric or polymeric materials. Fluid conduit 936 may connect components of the NWPT apparatus by conventional air-tight means such as friction fit, bayonet coupling, or barbed connectors. The conduit connections may be made permanent, or alternatively a quick-disconnect or other releasable means may be used to provide some adjustment flexibility to the apparatus 910.

Collection canister 940 may comprise any container suitable for containing wound fluids. For example, a rigid bottle may be used as shown or alternatively a flexible polymeric pouch may be appropriate. Collection canister 940 may contain an absorbent material to consolidate or contain the wound drainage or debris. For example, super absorbent polymers (SAP), silica gel, sodium polyacrylate, potassium polyacrylamide or related compounds may be provided within canister 940. At least a portion of canister 940 may be transparent to assist in evaluating the color, quality or quantity of wound exudates. A transparent canister may thus assist in determining the remaining capacity of the canister or when the canister should be replaced.

Leading from collection canister 940 is another section of fluid conduit 936 providing fluid communication with vacuum source 950. Vacuum source 950 generates or otherwise provides a negative pressure to the NWPT apparatus 910. Vacuum source 950 may comprise a peristaltic pump, a diaphragmatic pump or other mechanism that is biocompatible and draws fluids, e.g. atmospheric gasses and wound exudates, from the reservoir 914 appropriate to stimulate healing of the wound “w₁.” Preferably, the vacuum source 950 is adapted to produce a sub-atmospheric pressure in the reservoir 914 ranging between about 20 mmHg and about 500 mm Hg, about 75 mm Hg to about 125 mm Hg, or, more preferably, between about 40 mm HG and 80 mm Hg. Referring now to FIG. 7, a composite wound dressing and delivery apparatus 100 includes three distinct layers. Centrally located is the dressing layer 102 that may be used to form cover layer 934. Dressing layer 102 is interposed between a backing layer 104 and a delivery layer 106. Each of the three distinct layers 102, 104, and 106 is generally transparent to facilitate placement of the dressing layer 102 over a wound.

Dressing layer 102 may be formed from a variety of thin, transparent, polymeric membranes, such as polyurethane, elastomeric polyester or polyethylene. The thickness of the dressing layer 102 may, for example, be in the range of about 0.8 mils to about 1.2 mils. Thicknesses in this range may permit dressing layer 102 to conform to the contours of a patient's skin surrounding a wound, and accommodate evacuation cycles associated with an NWPT procedure. While the dressing layer 102 may be manufactured in any desired size or shape, the particular geometry of the wound to be treated may prompt customization of each individual dressing layer 102. As provided, dressing layer 102 is generally rectangular having a length of about 6 inches and a width of about 4 inches.

Dressing layer 102 has a lower surface 108 and an upper surface 110. Lower surface 108 is coated with an adhesive to facilitate adherence of the dressing layer 102 to the healthy skin “s₁” surrounding the wound “w₁.” The adhesive coating should provide firm, continuous adhesion to the skin “s₁” such that leak paths are not readily formed between the dressing layer 102 and the skin “s₁” when as reservoir 914 is subjected to the evacuation cycles of an NWPT treatment. The adhesive should also not unduly interfere with the transparency of dressing layer 102, and should peel away from the skin easily when the dressing layer 102 is no longer required.

The adhesive coating also preferably does not interfere with the transmission of moisture vapor through dressing layer 102. To promote enhanced moisture vapor transmission rate (MVTR) of the dressing layer 102, the adhesive coating may be interrupted in some embodiments such that only a periphery of dressing layer 102 is coated to form a seal with the skin “s₁” leaving a central portion of the dressing layer 102 uncoated. This arrangement is not necessarily preferred since cover layer 102 may be customized to accommodate the particular geometry of an individual wound and an appropriate periphery may not be known at the time of manufacturing. An adhesive coated substantially over the entire lower surface 108 may be selected that exhibits an MVTR equal to that of the film material.

Centrally located on the dressing layer 102 is a vacuum port 112 to facilitate connection to a vacuum tube 38. Vacuum port 112 is depicted schematically and may assume a variety of forms. For example, a structure similar to vacuum port 930 having a flange pre-affixed to dressing layer 102 may be provided along with the composite dressing 100. Alternatively, the vacuum port 112 may consist essentially of a pre-cut hole in the dressing layer 102, or in other embodiments, vacuum port 112 may comprise a marking to indicate a central location of the dressing layer 102 in which an opening may be cut by a clinician after dressing layer 102 is applied over a wound “w₁.”

Backing layer 104 is generally transparent and has a firm but releasable affinity for the adhesively coated lower surface 108 of dressing layer 102. Backing layer 104 covers the lower surface 104 and includes a peripheral region 114 that extends substantially beyond at least one edge of the dressing layer 102. Peripheral region 114 thus provides a gripping surface to facilitate the separation of the backing layer 112 from the dressing layer 102. Peripheral region 114 includes an indicator, such as first numerical indicator 116, printed or otherwise applied thereto. First numerical indicator 116 provides a prominent visual queue to indicate the order in which the three distinct layers 102, 104 and 106 should be separated.

Opposite peripheral region 114, backing layer 104 includes a background region 118 upon which a solid stripe is printed. Background region 118 is less translucent than dressing layer 102 and may be substantially opaque. Also printed on backing layer 104 is a circular reference 122, which is centrally located as to correspond with the location of vacuum port 112 on dressing layer 102. Surrounding the circular reference 122, a targeting grid 126 is printed or otherwise applied with regularly spaced reference lines in two orthogonal directions. Targeting grid 126 may be used to facilitate placement of the vacuum port 108 centrally over a wound “w₁” by providing a reference for measurement of the wound “w₁,” and by providing a reference for precise cutting or customization of the dressing layer 102.

Delivery layer 106 is adhered to the upper surface 110 of the dressing layer 102 in a releasable manner. Delivery layer 106 is substantially rigid in relation to dressing layer 102 to maintain the dressing layer 102 in a relatively smooth and unwrinkled configuration while the dressing layer 102 is applied to the skin “s₁.” Delivery layer 106 is, however, sufficiently flexible to conform to irregular contours of the skin “s₁” such that the dressing layer 102 may be pressed onto the skin “s₁” to form a substantially fluid tight seal therewith.

Preferably, both delivery layer 106 and upper surface 110 are non-adhesive, and may be adhered by heat lamination contact or similar means. A peripheral region 130 of delivery layer 106 overlies dressing layer 102, but is not adhered to dressing layer 102. Peripheral region 130 thus provides a gripping surface to facilitate separation of the delivery layer 106 from the dressing layer 102.

An indicator such as second numerical indicator 132 is positioned on the peripheral region 130 to indicate the order in which the three distinct layers 102, 104 and 106 should be separated. Second numerical indicator 132 is defined by the transparent or relatively transparent text and graphics surrounded by a darker background area of peripheral region 130. The background area of peripheral region 130 may be printed to have an appearance that is substantially similar to the appearance of background region 118 on backing layer 104. In this way, second numerical indicator 132 may be camouflaged or obscured when the backing layer 104 is adhered to the dressing layer 102 and revealed when backing layer 104 is separated from the dressing layer 102.

Delivery layer 106 also includes a central opening 134 to accommodate vacuum port 112 on the dressing layer 102 and a printed boundary 136 opposite peripheral region 130. Printed boundary 136 may be coincident with an edge of the dressing layer 102 to help identify the edge when the delivery layer 106 is adhered to the dressing layer 102.

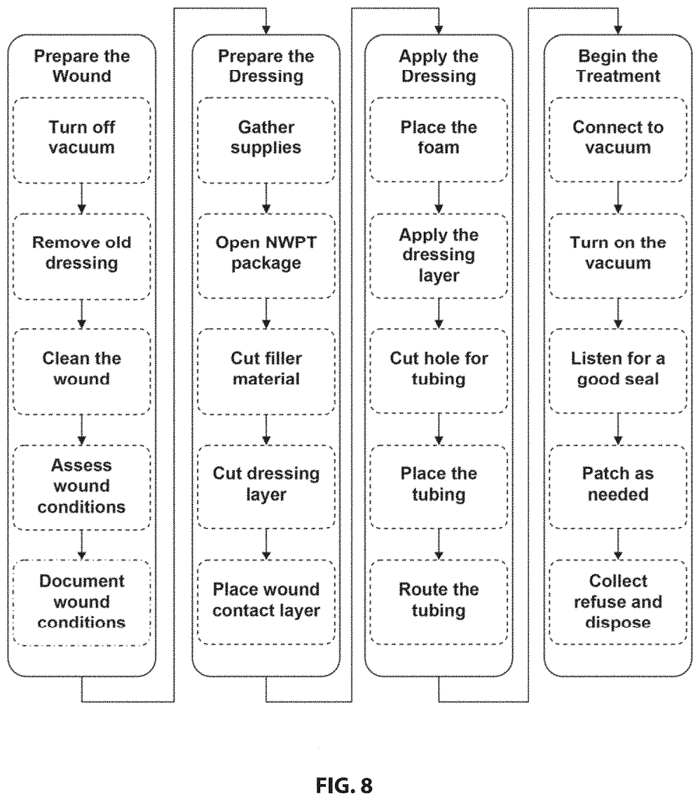

Referring now to FIG. 8, a process is described for changing a dressing 112 of an NWPT apparatus 910, as depicted in FIG. 6. The process makes use of a composite dressing and delivery apparatus 100 as depicted in FIG. 7, or any of the alternative composite apparatuses discussed below. The procedure includes four major steps, each including a number of sub-steps. Each of the steps and sub-steps may be performed in any suitable order including the order depicted.

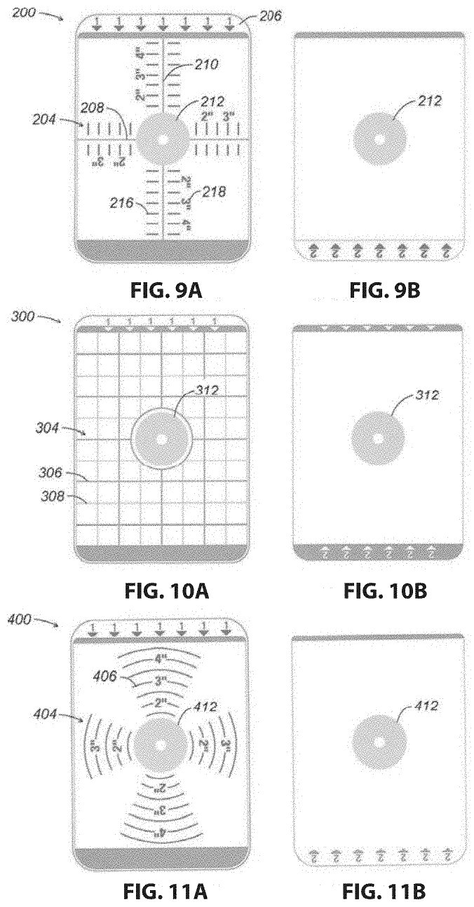

In the first major step, the wound “w₁” is prepared. The vacuum source 950 may be deactivated and the existing dressing 912 may be removed. The wound “w₁” may be cleaned and wound conditions may then be assessed. The sub-step of documenting the wound conditions may be performed concurrently with the second major step of preparing the dressing, and may be facilitated by any of the composite wound dressing and delivery apparatuses depicted in FIG. 9A through FIG. 11B.

FIG. 9A depicts a composite wound dressing and delivery apparatus 200 having a Cartesian targeting grid 204 printed on a backing layer 206 thereof to assist in documentation of wound conditions. Cartesian targeting grid 204 includes a horizontal axis 208 and an orthogonal vertical axis 210 extending through a central location corresponding with the location of vacuum port 212. Rule marks 216 flank the horizontal and vertical axes 208, 210 in a regularly spaced intervals preferably corresponding to the units of a standard measurement system, e.g. metric or English. Numerical markers 218 may correspond to a number of inches that is twice the distance from a center of vacuum port 212. Composite wound dressing and delivery apparatus 200 may be placed over a wound “w₁” such that vacuum port 212 is centrally located, and a distance across the wound along in two axes may be noted and documented.

FIG. 10A and FIG. 11A depict alternate composite dressing and delivery apparatuses 300, 400, which may be used in a similar manner to document the size of a wound “w₁.” Composite apparatus 300 includes a targeting grid 304 similar to targeting grid 126 discussed above with reference to FIG. 7, Targeting grid 304 includes major gridlines 306 which are adapted to appear more prominent than minor gridlines 308. Each of the grid lines 306, 308 may correspond to a predetermined unit of measurement and distance from vacuum port 312. Composite system 400 includes a polar or radial targeting grid 404. Radial targeting grid 404 includes curvilinear rule lines 406 arranged around vacuum port 412.

In the second major step, the dressing is prepared. Once supplies have been gathered, an NWPT package (not shown) may be opened. An NWPT package may be provided that includes a sterilized kit including various items used in an NWPT procedure such as a composite wound dressing and delivery apparatus 100, material for wound contact layer 918, material for filler 920 and other items including those described herein below. Once the NWPT package is opened, the packaging material may be used as a clean preparation surface for inventory and organization of the kit components. The packaging material should therefore exhibit a tendency to lie flat and should be sufficient in size to accommodate each of the kit components thereon.

Material may be provided in an NWPT kit for filler 920. The material may be cut to size to allow filler 920 to fill or overfill the wound “w₁” as described above. The composite system 100 may then be cut appropriate the size of the wound “w₁.” To allow dressing layer 102 to form an appropriate seal with the skin “s₁,” composite system 100 should be cut to permit from about one inch to about one and one half inches of contact between the skin “s₁” and the adhesively coated lower surface 108 of dressing layer 102 around the wound “w₁.” Cutting the composite system 100 may be facilitated by the targeting grid 126, which provides reference to guide the cut.

Surgical scissors (not shown), may be used to make the cut and may be sterilized or cleaned prior to each use. The scissors need not be included in the NWPT kit. The scissors may also be used to cut wound contact layer 918 to size before it is placed adjacent to the wound “w₁.”

In the third major step, the dressing may be applied to the wound “w₁.” The filler 920 may be placed over the contact layer 918. Often, a portion dressing layer 102 that was cut from composite system 100 in a previous step is used to tack the filler 920 in place.

Next, dressing layer 102 may be applied over the wound “w₁.” The backing layer 104 is first separated to expose the adhesive coating on the lower surface 108 of dressing layer 102. First numerical indicator 116 indicates that the peripheral region 114 may first be grasped to remove the backing layer 104. Once the adhesive is exposed, dressing layer 102 may be pressed onto the skin “s₁” to form a fluid-tight seal therewith. With backing layer 104 removed, second numerical indicator 132 is revealed as described above. The alternate embodiments depicted in FIGS. 9B, 10B and 11B demonstrate other arrangements that may permit a second numerical indicator to be revealed upon the removal of the respective backing layer. Second numerical indicator 132 indicates that the delivery layer 106 may be separated from dressing layer 102 once the dressing layer 102 has been placed over the wound “w₁.” The delivery layer 106 should readily separate from the upper surface 110 of the dressing layer 102 such that the seal formed around the wound “w₁” is not disturbed and so as not to cause the patient any undue pain.

If necessary, a hole may be cut in vacuum port 112 to receive fluid conduit 936. Fluid conduit 936 may be placed relative to vacuum port 112 such that the fluid conduit 936 may communicate with reservoir 914. Next, an exposed portion of fluid conduit may be oriented or routed so as not to interfere with patient movement or comfort. Again, a portion of the dressing layer 102 that was cut from composite system 100 in a previous step may be used to secure the fluid conduit 936.

In the fourth major step, treatment of the wound “w₁” may begin. The fluid conduit 936 may be connected to vacuum source 950 through canister 940. The vacuum source 99 may then be activated to evacuate atmospheric gasses from the reservoir 914. A distinctive sound or audible indicator may indicate whether a proper seal has been achieved over the wound “w₁.” If necessary, any leaks identified may be patched with a portion of the dressing layer 102 that was cut from composite system 100 in a previous step.

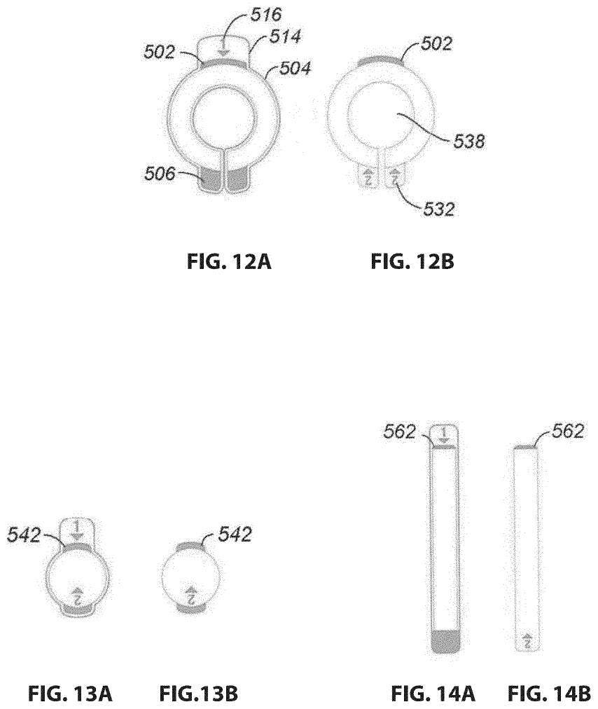

Alternatively, a prefabricated patch, such as the patches depicted in FIG. 12A through FIG. 14B, may be used to patch any leaks identified. Each of the patches depicted may be formed from materials similar to those selected for dressing layer 102, and may be provided with a composite delivery apparatus similar to the delivery apparatus provided for dressing layer 102. For example, a patch layer 502, as depicted in FIGS. 12A and 12B, includes a backing layer 504 and a delivery layer 506. Backing layer 504 includes a peripheral region 514 extending beyond an edge of the patch 502. First numerical indicator 516 indicates that peripheral region 514 may be grasped to remove the backing layer 504 and expose an adhesive coating on an underside of patch 502. A second numerical indicator 532 (FIG. 12B) is also thereby revealed. The patch layer 502 may be applied over a leak, and delivery layer 506 may be removed.

Patch layer 502 is shaped such that an opening or interior region 538 has a geometry that is substantially similar to the geometry of the vacuum port 112. Interior region 538 may be open to receive vacuum port 112 therein. Creating a seal around vacuum port 112 may present a challenge, and incorporating a patch layer 502 configured to approximate the particular size and shape of the perimeter of vacuum port 112 into an NWPT kit may be helpful. Opening 538 is substantially circular to accommodate vacuum port 112, but other configurations may be used. For example, a patch (not shown) having a semicircular or other arc shape may be provided.

Patch 542 depicted in FIGS. 13A and 13B has a substantially round geometry and may be provided to patch small leaks in anywhere on the dressing layer 102. Patch 562 depicted in FIGS. 14A and 14B has an elongate geometry and may be provided to patch a leak along an edge of dressing layer 102.

Each of the patches described above may be provided as a kit component in an NWPT package. Having prefabricated patches on hand can ensure the integrity of a fluid tight seal over a wound “w₁.” Also prefabricated patches may be used to tack filler 920 in place in a sterile manner, or to conveniently secure the position of fluid conduit 38. Prefabricated patches may be provided to serve any function that may otherwise be served by a portion of the dressing layer 102 that was cut from composite system 100.

A final sub-step may be to collect and retain any unused components, e.g., patches or portions of dressing layer 102, and to dispose of any refuse. Backing layer 104 and delivery layer 106 each include printing thereon, e.g., the targeting grid 126 or numerical identifiers 116, 132, which can assist in locating these components for disposal.

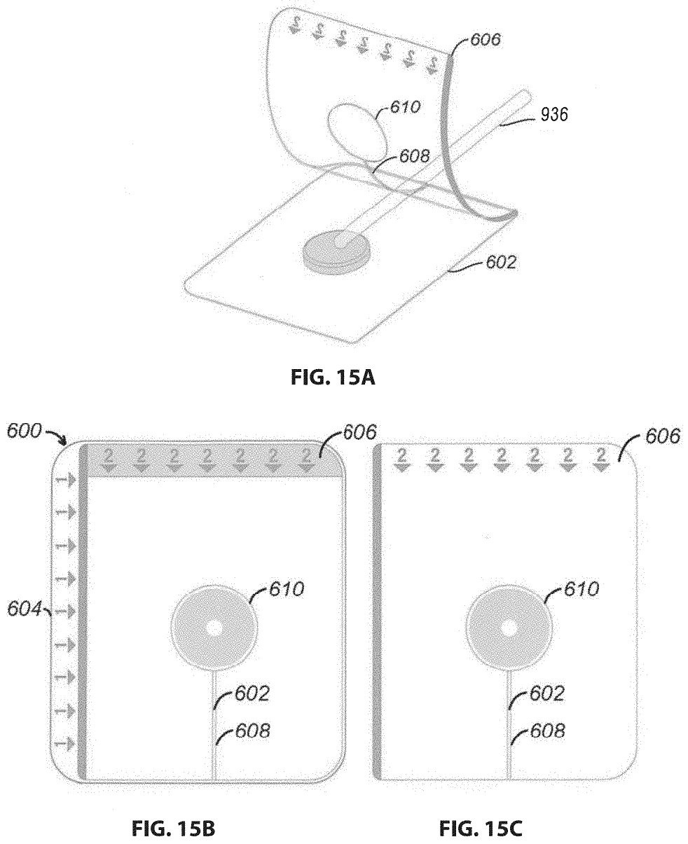

Referring now to FIG. 15A through FIG. 15C, an alternate embodiment of a composite dressing and delivery apparatus is depicted generally as 600. Composite system 600 includes a dressing layer 602, a backing layer 604 and a delivery layer 606. Delivery layer 606 includes a narrow slit 608 therein extending from central opening 610 to an exterior edge to allow the delivery layer 606 to be conveniently removed after fluid conduit 936 has been routed as depicted in FIG. 11A.

Backing layer 604 includes a peripheral region 614 with a first numerical indicator 616, and a background region 618 positioned along an edge orthogonal to the peripheral region 614. Delivery layer 606 includes a second numerical indicator 632 printed in a darker color than background region 618. Second numerical indicator 632 is thus visible when backing layer 604 is adhered to dressing layer 602, but more prominent when backing layer 604 is removed,

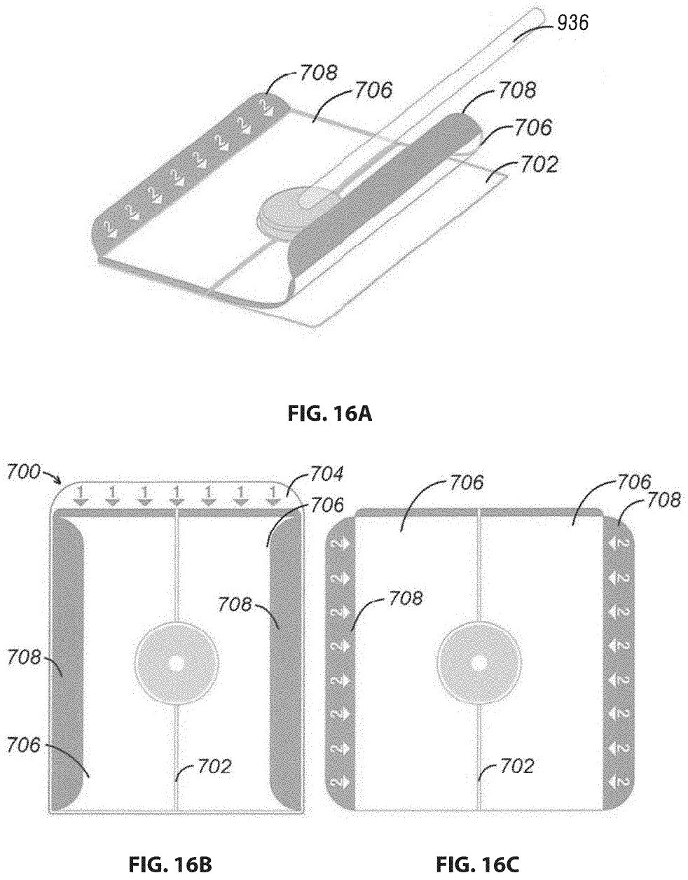

Referring now to FIG. 16A through FIG. 16C, an alternate embodiment of a composite dressing and delivery apparatus is depicted generally as 700. Composite system 700 includes a dressing layer 702, a backing layer 704 and a pair of opposed delivery layers 706. Each delivery layer 706 includes a tab 708 along an edge thereof that protrudes beyond the extents of dressing layer 702. As provided tabs 708 may be folded inwardly as depicted in FIG. 16B. In use, each tab 708 may be folded out to provide a non-adhesive gripping surface that may be handled to place the dressing layer 702 over the wound. Tabs 708 may eliminate a need for gripping an adhesive coating on dressing layer 702, and thereby improve the sealing characteristics of the dressing layer 702 and promote the cleanliness of the wound “w₁.”

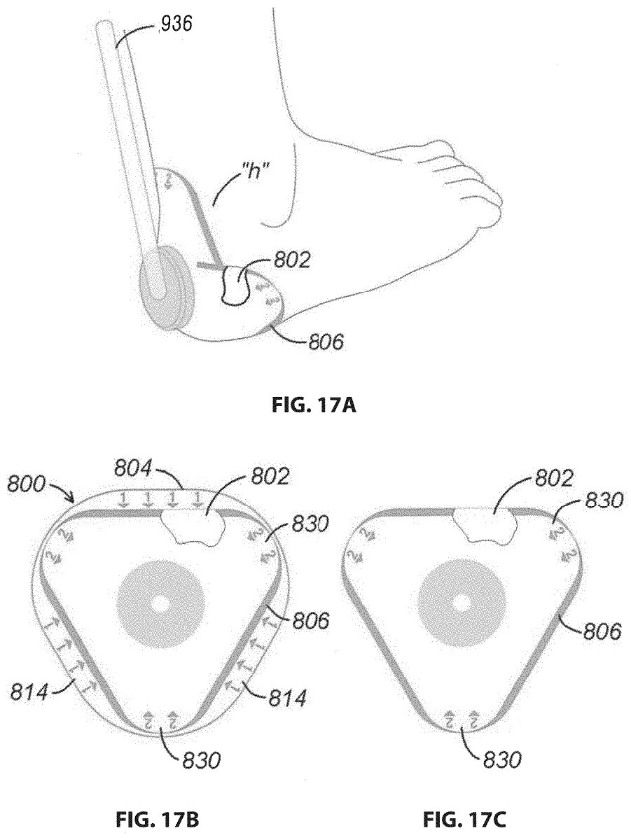

Referring now to FIG. 17A through FIG. 17C, another embodiment of a composite dressing and delivery apparatus is depicted generally as 800. Composite system 800 includes a dressing layer 802, a backing layer 804 and a delivery layer 806. Dressing layer 802 is generally triangular in shape such that it may conform to contours of an irregular body pan such as a heel “h” as depicted in FIG. 17A. Also, a dressing layer 802 configured with a triangular shape may offer a convenient form to include multiple peripheral regions 814 on backing layer 804, and multiple peripheral regions 830 on delivery layer 806. Multiple peripheral regions 814, 830 may facilitate placement of dressing layer 802 in the event that customizing or cutting the composite system 800 results in the removal of one or more of the peripheral regions 814, 830.

### System for Providing Continual Drainage in Negative Pressure Wound Therapy

A system for subatmospheric pressure therapy in connection with healing a wound is provided. The system includes a wound dressing cover dimensioned for positioning relative to a wound bed of a subject to establish a reservoir over the wound bed in which subatmospheric pressure may be maintained, a subatmospheric pressure mechanism including, a housing, a vacuum source disposed in the housing, and a collection canister in fluid communication with the vacuum source. The system further includes an exudate conduit in fluid communication with the wound dressing and the collection canister for collecting exudate removed from the reservoir and deposited in the collection canister under influence of the vacuum source and a vent conduit in fluid communication with the collection canister and the wound dressing for introducing air into the reservoir to facilitate flow of exudate through the exudate conduit.

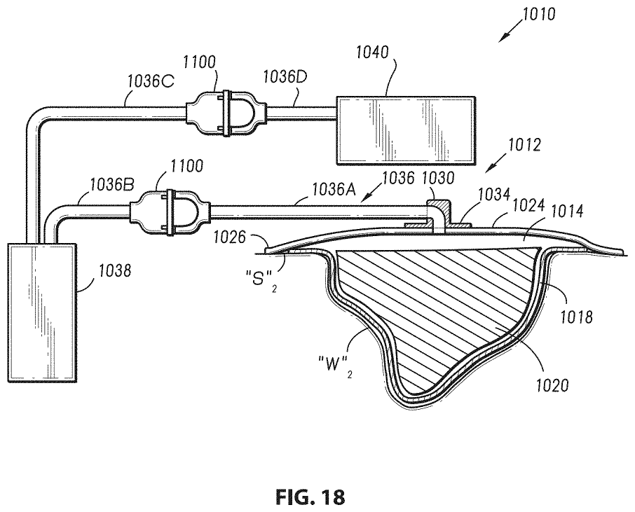

Referring to FIG. 18, a NPWT apparatus according to an embodiment of the present disclosure is depicted generally as 1010 for use on a wound bed “w₂” surrounded by healthy skin “s₂”. Negative pressure wound therapy apparatus 1010 includes a wound dressing 1012 positioned relative to the wound bed “w₂” to define a vacuum chamber 1014 about the wound bed “w₂” to maintain negative pressure at the wound area. Wound dressing 1012 includes a contact layer 1018, a wound filler 1020 and a wound cover 1024.

Contact layer 1018 is intended for placement within the wound bed “w₂” and may be relatively non-supportive or flexible to substantially conform to the topography of the wound bed “w₂”. A variety of materials may be used for the contact layer 1018. Contact layer 1018 selection may depend on various factors such as the patient's condition, the condition of the periwound skin, the amount of exudate and/or the condition of the wound bed “w₂”. Contact layer 1018 may be formed from perforated film material. The porous characteristic of the contact layer 1018 permits exudate to pass from the wound bed “w₂” through the contact layer 1018. Passage of wound exudate through the contact layer 1018 may be substantially unidirectional such that exudate does not tend to flow back into the wound bed “w₂”. Unidirectional flow may be encouraged by directional apertures, e.g., apertures positioned at peaks of undulations or cone-shaped formations protruding from the contact layer 1018. Unidirectional flow may also be encouraged by laminating the contact layer 1018 with materials having absorption properties differing from those of the contact layer 1018, or by selection of materials that promote directional flow. A non-adherent material may be selected for forming the contact layer 1018 such that the contact layer 1018 does not tend to cling to the wound bed “w₂” or surrounding tissue when it is removed. One example of a material that may be suitable for use as a contact layer 1018 is commercially available under the trademark XEROFLOW® offered by Tyco Healthcare Group LP (d/b/a Covidien). Another example of a material that may be suitable for use as the contact layer 1018 is the commercially available CURITY® non-adherent dressing offered by Tyco Healthcare Group LP (d/b/a Covidien).

Wound filler 1020 is positioned in the wound bed “w₂” over the contact layer 1018 and is intended to transfer wound exudate. Wound filler 1020 is conformable to assume the shape of any wound bed “w₂” and may be packed up to any level, e.g., up to the level of healthy skin “s₂” or to overfill the wound such that wound filler 1020 protrudes over healthy skin “s₂”. Wound filler 1020 may be treated with agents such as polyhexamethylene biguanide (PHMB) to decrease the incidence of infection and/or other medicaments to promote wound healing. A variety of materials may be used for the wound filler 1020. An example of a material that may be suitable for use as the wound filler is the antimicrobial dressing commercially available under the trademark KERLIX™ AMD™ offered by Tyco Healthcare Group LP (d/b/a Covidien).

Cover layer 1024 may be formed of a flexible membrane, e.g., a polymeric or elastomeric film, which may include a biocompatible adhesive on at least a portion of the cover layer 1024, e.g., at the periphery 1026 of the cover layer 1024. Alternately, the cover layer 1024 may be a substantially rigid member. Cover layer 1024 may be positioned over the wound bed “w₂” such that a substantially continuous band of a biocompatible adhesive at the periphery 1026 of the cover layer 1024 forms a substantially fluid-tight seal with the surrounding skin An example of a material that may be suitable for use as the cover layer 1024 is commercially available under the trademark CURAFORM ISLAND® offered by Tyco Healthcare Group LP (d/b/a Covidien).

Cover layer 1024 may act as both a microbial barrier and a fluid barrier to prevent contaminants from entering the wound bed “w₂” and to help maintain the integrity thereof.

In one embodiment, the cover layer 1024 is formed from a moisture vapor permeable membrane, e.g., to promote the exchange of oxygen and moisture between the wound bed “w₂” and the atmosphere. An example of a membrane that may provide a suitable moisture vapor transmission rate (MVTR) is a transparent membrane commercially available under the trade name POLYSKIN® II offered by Tyco Healthcare Group LP (d/b/a Covidien). A transparent membrane may help to permit a visual assessment of wound conditions to be made without requiring removal of the cover layer 1024.

Wound dressing 1012 may include a vacuum port 1030 having a flange 1034 to facilitate connection of the vacuum chamber 1014 to a vacuum system. Vacuum port 1030 may be configured as a rigid or flexible, low-profile component and may be adapted to receive a conduit in a releasable and fluid-tight manner. An adhesive on at least a portion of the underside of the flange 1034 may be used to provide a mechanism for affixing the vacuum port 1030 to the cover layer 1024. The relative positions, size and/or shape of the vacuum port 1030 and the flange 1034 may be varied from an embodiment depicted in FIG. 18. For example, the flange 1034 may be positioned within the vacuum chamber 1014 such that an adhesive on at least a portion of an upper side surface of the flange 1034 affixes the vacuum port 1030 to the cover layer 1024. A hollow interior portion of the vacuum port 1030 provides fluid communication between the conduit 1036 and the vacuum chamber 1014. Conduit 1036 extends from the vacuum port 1030 to provide fluid communication between the vacuum chamber 1014 and the vacuum source 1040. Alternately, the vacuum port 1030 may not be included in the dressing 1012 if other provisions are made for providing fluid communication with the conduit 1036.

Any suitable conduit may be used for the conduit 1036, including conduit fabricated from flexible elastomeric or polymeric materials. In the negative pressure wound therapy apparatus 1010 illustrated in FIG. 18, the conduit 1036 includes a first conduit section 1036A, a second conduit section 1036B, a third conduit section 1036C and a fourth conduit section 1036D. The first conduit section 1036A extends from the vacuum port 1030 and is coupled via a fluid line coupling 1100 to the second conduit section 1036B, which extends to the collection canister 1038. The third conduit section 1036C extends from the collection canister 1038 and is coupled via another fluid line coupling 1100 to the fourth conduit section 1036D, which extends to the vacuum source 1040. The shape, size and/or number of conduit sections of the conduit 1036 may be varied from the first, second, third and fourth conduit sections 1036A, 1036B, 1036C and 1036D depicted in FIG. 18.

The first, second, third and fourth conduit sections 1036A, 1036B, 1036C and 1036D of the conduit 1036 may be connected to components of the apparatus 1010 by conventional air-tight means, such as, for example, friction fit, bayonet coupling, or barbed connectors. The connections may be made permanent. Alternately, a quick-disconnect or other releasable connection means may be used to provide some adjustment flexibility to the apparatus 1010.

Collection canister 1038 may be formed of any type of container that is suitable for containing wound fluids. For example, a semi-rigid plastic bottle may be used for the collection canister 1038. A flexible polymeric pouch or other hollow container body may be used for the collection canister 1038. Collection canister 1038 may contain an absorbent material to consolidate or contain the wound fluids or debris. For example, super absorbent polymers (SAP), silica gel, sodium polyacrylate, potassium polyacrylamide or related compounds may be provided within collection canister 1038. At least a portion of canister 1038 may be transparent or semi-transparent, e.g., to permit a visual assessment of the wound exudate to assist in evaluating the color, quality and/or quantity of exudate. A transparent or semi-transparent portion of the collection canister 1038 may permit a visual assessment to assist in determining the remaining capacity or open volume of the canister and/or may assist in determining whether to replace the collection canister 1038.

The collection canister 1038 is in fluid communication with the wound dressing 1012 via the first and second conduit sections 1036A, 1036B. The third and fourth conduit sections 1036C, 1036D connect the collection canister 1038 to the vacuum source 1040 that generates or otherwise provides a negative pressure to the collection canister 1038. Vacuum source 1040 may include a peristaltic pump, a diaphragmatic pump or other suitable mechanism. Vacuum source 1040 may be a miniature pump or micropump that may be biocompatible and adapted to maintain or draw adequate and therapeutic vacuum levels. The vacuum level of subatmospheric pressure achieved may be in the range of about 20 mmHg to about 500 mmHg. In embodiments, the vacuum level may be about 75 mmHg to about 125 mmHg, or about 40 mmHg to about 80 mmHg. One example of a peristaltic pump that may be used as the vacuum source 1040 is the commercially available Kangaroo PET Eternal Feeding Pump offered by Tyco Healthcare Group LP (d/b/a Covidien). Vacuum source 1040 may be actuated by an actuator (not shown) which may be any means known by those skilled in the art, including, for example, alternating current (AC) motors, direct current (DC) motors, voice coil actuators, solenoids, and the like. The actuator may be incorporated within the vacuum source 1040.

In embodiments, the negative pressure wound therapy apparatus 1010 includes one or more fluid line couplings 1100 that allow for selectable coupling and decoupling of conduit sections. For example, a fluid line coupling 1100 may be used to maintain fluid communication between the first and second conduit sections 1036A, 1036B when engaged, and may interrupt fluid flow between the first and second conduit sections 1036A, 1036B when disengaged. Thus, fluid line coupling 1100 may facilitate the connection, disconnection or maintenance of components of the negative pressure wound therapy apparatus 1010, including the replacement of the collection canister 1038. Additional or alternate placement of one or more fluid line couplings 1100 at any location in line with the conduit 1036 may facilitate other procedures. For example, the placement of a fluid line coupling 1100 between the third and fourth conduit sections 1036C, 1036D, as depicted in FIG. 18, may facilitate servicing of the vacuum source 1040.

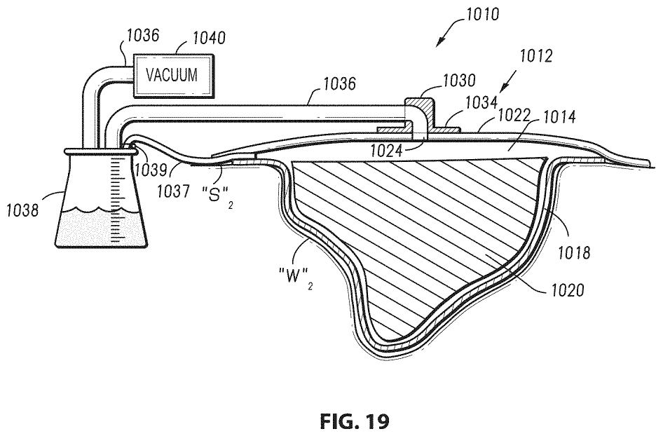

Referring to FIG. 19, an NPWT apparatus similar to the NPWT apparatus of FIG. 18 is depicted generally as 1010 for use on a wound “w₂” surrounded by healthy skin “s₂.” The NPWT apparatus 1010 of FIG. 19 includes a vent conduit 1037 that extends from between contact layer 1018 and cover layer 1022 of wound dressing 1012 to collection canister 1038. Vent conduit 1037 may be integral formed with wound dressing 1012. Alternatively, vent conduit 1037 may be inserted between contact layer 1018 and cover layer 1022 by a clinician during application of the wound dressing 1012, or may have been previously inserted therebetween prior to application. Vent conduit 1037 may be releasably connected to the collection canister 1038 by conventional air-tight means such as friction fit, bayonet coupling, or barbed connectors.

Vent conduit 1037 is configured to provide a low flow of air from the reservoir 1014 to the collection canister 1038. Vent conduit 1037 includes a smaller diameter than exudate conduit 1036 and may be formed of any suitable conduit including those fabricated from flexible elastomeric or polymeric materials. An air filter 1039 positioned along the air flow path filters the air flowing from collection canister 1038 to remove any impurities, including bacteria and other infectious material. Filter 1039 may include a hydrophobic material to prevent wetting.

In operation, wound dressing 1012 is placed adjacent a wound “w₂” with the vent conduit 1037 extending from between the contact layer 1018 and the cover layer 1022. If the vent conduit 1037 is not integral formed with the wound dressing 1012, the clinician may be required to position the vent conduit 1037 between the layers during application of the wound dressing 1012. Vacuum source 50 is then activated to produce a sub-atmospheric pressure in the reservoir 1014 of the wound dressing 1012. Fluid from the reservoir 1014 is drawn through aperture 1024 in cover layer into fluid port 1030 and along exudate conduit 1036 to be deposited in collection canister 1040 As fluid and other exudates are drawn through exudate conduit 1036, filtered air is received within the reservoir 1014 of the wound dressing 1012 through the vent conduit 1037. The low flow filtered air flowing from the collection canister 1038 through the vent conduit 1037, in combination with the high flow drainage occurring through exudate conduit 1036, creates a sump action between the reservoir 1014 and the collection canister 1040. This sump action ensures continuous flow through exudate conduit 1036, thereby preventing fluid stagnation and its complications. Because of capillary action, fluid from reservoir 1014 only flows through the larger diameter exudate conduit 1036.

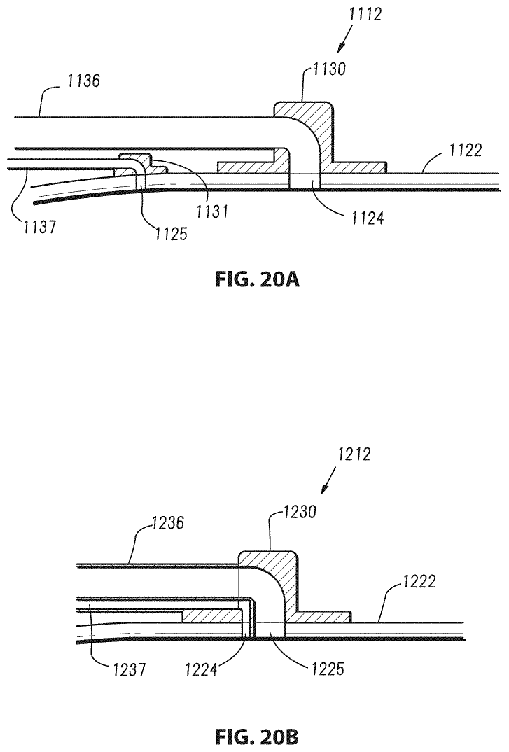

With reference now to FIG. 20A, in an alternative embodiment of the present disclosure, a wound dressing 1112 is substantially similar to wound dressing 1012 described hereinabove, and will only be described as relates to the differences therebetween. Wound dressing 1112 includes a cover layer 1122 having a first or fluid aperture 1124 and a second or vent aperture 1125. A fluid port 1130 is in fluid communication with fluid aperture 1124 and is configured for operable engagement with exudate conduit 1136. A vent port 1131 is in fluid communication with vent aperture 1125 and is configured for operable engagement with vent conduit 1137. Fluid and vent ports 1130, 1131 may be affixed to cover layer 1122 in any suitable manner. Each of fluid and vent port 1130, 1131 are in fluid communication with collection canister 1038 (FIGS. 18 and 19).

Wound dressing 1112 operates in substantially the same manner as wound dressing 1012. When connected to collection canister 1040 and the vacuum source 50 is activated, the sub-atmospheric pressure produced by the vacuum source 50 creates a suction that draws fluid from the reservoir Vent conduit provides the reservoir with a low flow of filtered air to ensure continuous fluid flow through the exudate conduit 1136.

Turning now to FIG. 20B, in another embodiment, wound dressing 1212 is substantially similar to the wound dressings 1012, 1112 described hereinabove. Wound dressing 1212 includes a cover layer 1222 having a first and second aperture 1224, 1225. Positioned adjacent first and second apertures 1224, 1225 is a fluid/vent port 1230. Port 1230 is configured to fluidly communicate first aperture 1224 of wound dressing 1212 with collection canister 1038 (FIGS. 18 and 19) via exudate conduit 1236. Port 1230 is further configured to fluidly communicate second aperture 1225 of wound dressing 1212 with collection canister 1040 via vent conduit 1237. As discussed above, the difference in size between exudate conduit 1236 and vent conduit 1237 results in capillary action that causes fluid to flow only through the larger exudate conduit 1036.

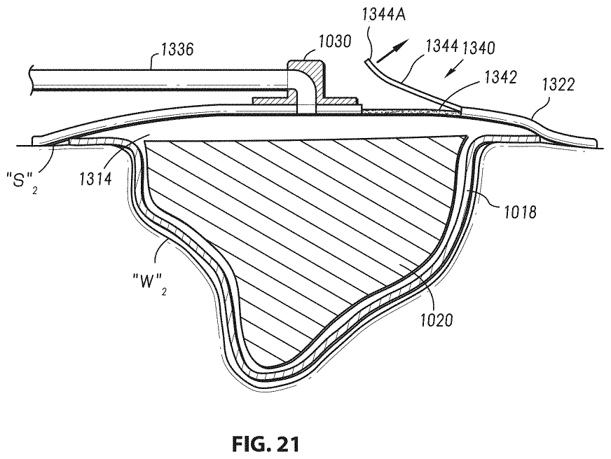

With reference now to FIG. 21, in yet another embodiment, a wound dressing 312 similar to those described above including a vent assembly 1340 formed in a cover layer 1322. Vent assembly 1340 includes a filter member 1342 and a flap or cover member 1344. Filter member 1342 may be integrally formed with, or otherwise affixed to, the cover layer 1322. In one embodiment, filter member 1342 is secured to the cover layer 1322 with an adhesive. Filter member 1342 is configured to provide reservoir 1314 of wound dressing 312 with filtered air. To prevent wetting, the filter member 1342 may be hydrophobic. Filter member 1342 may be sized depending on the desired flow therethrough. A larger filter member 1342 would provide a greater amount of airflow; however, if the filter member 1342 is too large, it may reduce the effectiveness of the NWPT.

Flap 1344 may be integrally formed with cover layer 1322. Alternatively, flap may be releasably secured over filter member 1342. Flap 1344 may be attached to or separable from cover member 1322. Flap 1344 may be configured to selectively partially or completely uncover filter member 1342. In this manner, a clinician may affect the flow of air into the reservoir 1314. Although shown including flap 1344, it is envisioned that wound dressing 312 may be provided with filter member 1342 exposed.

In use, wound dressing 312 is applied to a wound “w₂” in a conventional manner. Activation of the vacuum source 1040 (FIGS. 18 and 19) initiates drainage from reservoir 1314 of wound dressing 312. At any time prior to or during the drainage process, flap 1344 may be partially or complete pulled back to expose filter member 1342. As described above, the more of filter member 1342 that is exposed, the greater the possible airflow into reservoir 1014. The airflow provided to reservoir 1014 through filter member 1342 acts in a manner similar to the sump action described above. In this manner, vent assembly 1340 permits continuous fluid flow through exudate conduit 1336, thereby preventing fluid stagnation and its complications.

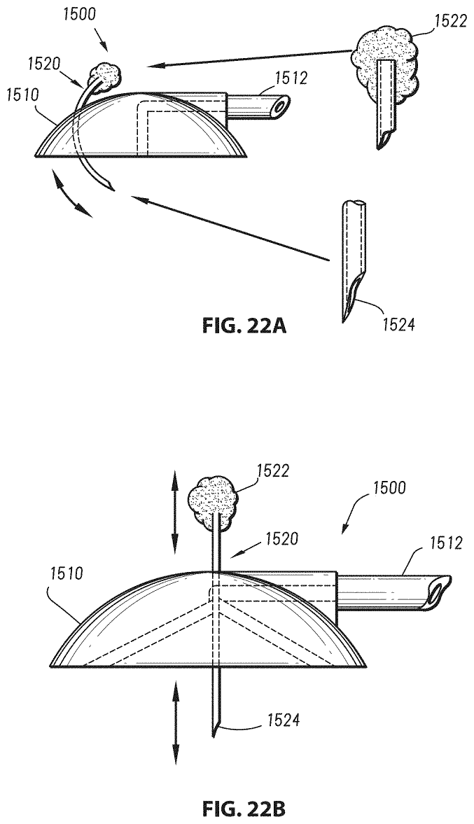

With reference to FIGS. 22A and 22B, in yet another embodiment, a wound port 1500 is shown. Wound port 1500 is suitable for use with the above described wound dressings. Wound port 1500 has a plastic cover 1510 which includes a vacuum port 1512. In addition to the vacuum port 1512, the plastic cover 1510 has tube 1520. Tube 1520 may be made of a small-bore stainless steel or rigid plastic. Tube 1520 is used to provide a controlled or fixed leak by admitting air into the wound dressing. Tube 1520 can be arranged to allow the insertion of tube 1520 into the wound port 1500 so that depth adjustment and placement within the wound packing material is possible as indicated by the arrows in 5A and 5B. As such, air can be injected into the wound packing material to direct movement of excess exudate toward the vacuum port and out of the wound. Tube 1520 may have a valve (not shown) to adjust the flow rate of air into the wound bed. The valve may be a small needle valve that can be attached to the tube 1520 to allow for infinite adjustment of air flow into the wound dressing.

The end of tube 1520 that may be exposed to ambient atmosphere or to a source of air may include a filter 1522. Filter 1522 may be a q-tip like air filter to prevent clogging of the tube and also prevent dirt and other contaminants from entering the wound site. Alternatively, filter 1522 may include a charcoal filter to prevent odor, a hydrophobic filter, or any sintered or porous material. The tip of tube 1520 that is inserted into the wound packing material may be equipped with a puncturing tip 1524 to allow for easier insertion into the wound packing material.

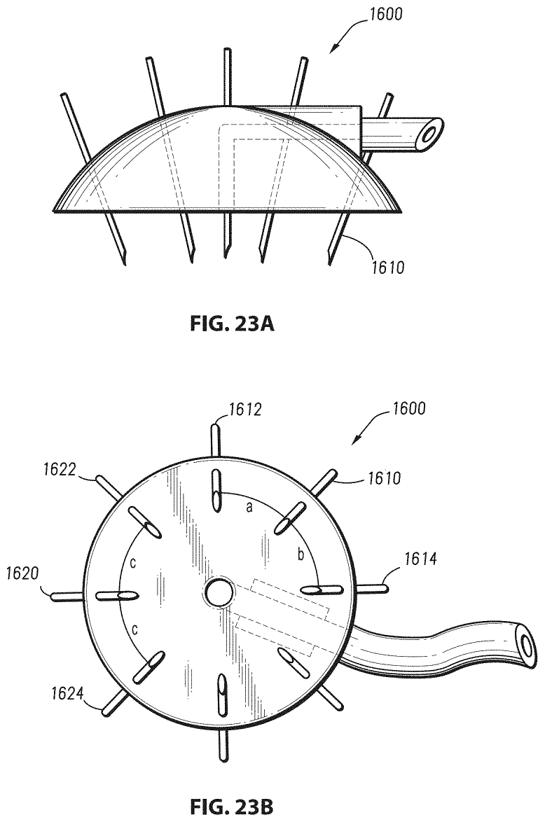

With reference to FIGS. 23A and 23B, in yet another embodiment, a wound port 1600 is shown. As shown in FIG. 23A, wound port 1600 has tube 1610 in separate locations around a circumference of the wound port 1600. Each tube may include a punctured tip or a filter as described above. As shown in FIG. 23B, the distance “a” between tube 1610 and tube 1612 may be one distance and the distance “b” between tube 1610 and 1614 may be a distance different the distance “a”. On the other hand, the difference between each tube may be similar as in the distance “c” between tube 1620 and 1622 and tube 1620 and 1624. Although FIGS. 23A and 23B show a specific number of tubes, any number of tubes may be arranged outside a circumference of the wound port 1600.

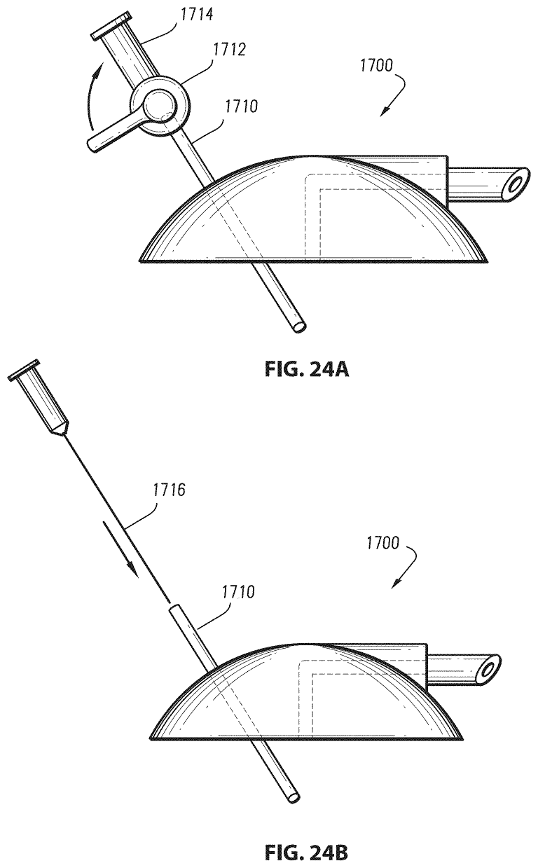

With reference to FIGS. 24A and 24B, in yet another embodiment, a wound port 1700 is shown having a tube 1710 which is similar to the tubes described above. Tube 1710 may be slightly larger in diameter to allow for fluids to enter the wound site. The fluids may include a solution to flush the wound such as saline or it may be an anesthetic to anesthetize the wound area. Tube 1710 may be fitted with valve 1712 to open and close the pathway into the wound site. Additionally, the end of tube 1710 may be fitted with a luer connector 1714 to create a fluid tight connection with additional tubing, syringes, or any other conduits. Alternatively, instead of a valve, a plug (not shown) could be used to close the luer connector. With reference to FIG. 24B, a hypodermic needle 1716 may be inserted into tube 1710. Hypodermic needle 1716 could be used to deliver a solution to a specific area of the wound or it could be used to obtain a sample of blood, exudate or tissue from the wound site.

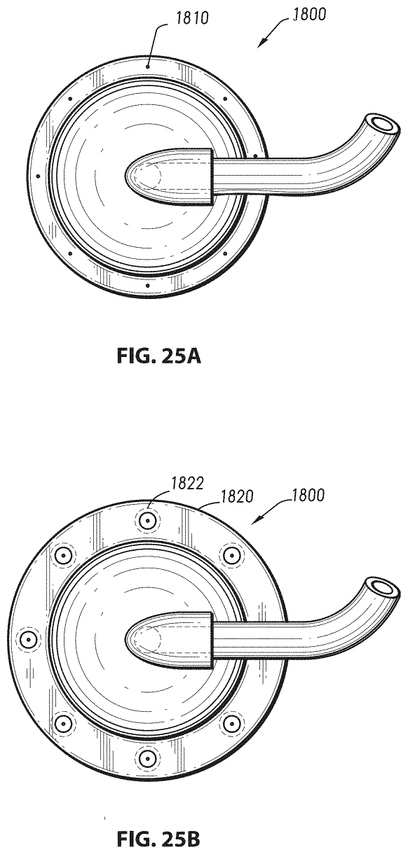

With reference to FIGS. 25A and 25B, in yet another embodiment, a wound port 1800 is shown. Instead of using tubes as described above to allow a controlled or fixed leak, a number of small holes arranged in a circumference around the wound port 1800 may be provided. The holes may take the form of a simple puncture 1810 of a given size as shown in FIG. 25A. Alternatively, the holes 1822 may be formed in a plate 1820 that is radio frequency (RF) welded to the wound port 1800.

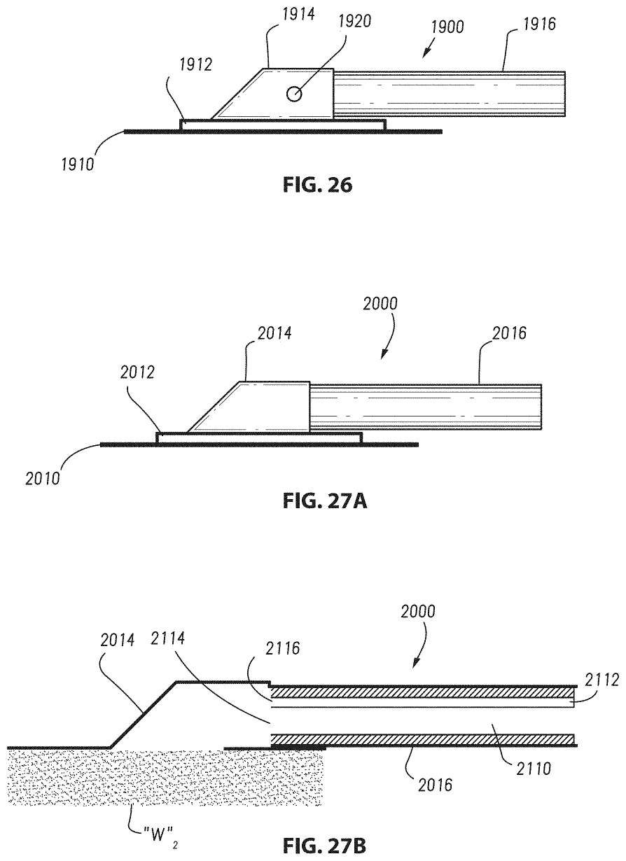

With reference to FIG. 26, in yet another embodiment, a cross section of wound port 1900 is shown. Wound port 1900 is operatively connected to wound dressing 1910 and includes a flange 1912. Flange 1912 may have a circular or any polygonal shape. A body 1914 is connected to the flange 1912 which is fluidly connected to conduit 1916. Conduit 1916 leads directly or indirectly to the collection canister. Body has as small orifice used to provide a controlled leak into the wound site. The diameter of the orifice 1920 and the pressure difference between the outside of the wound port 1900 and the inside of the wound port 90 create a controllable air leak into the wound port 1900 via the orifice. The small orifice 1920 can be created in various ways. The orifice 1920 can be integral to the port design, such as a molded in feature. It can be created via post molding micro-piercing into the port using a needle or syringe. Alternatively, assembly or insertion of a small tube that allows for communication of air from outside the wound port 1900 to inside the wound port 1900 can be used to create the orifice 1920.

With reference to FIGS. 27A and 27B, in yet another embodiment, a wound port 2000 is shown. Wound port 2000 is operatively connected to wound dressing 2010 and includes a flange 2012. Flange 2012 may have a circular or any polygonal shape. A body 2014 is connected to the flange 2012 which is fluidly connected to conduit 2016. Conduit 2016 leads directly or indirectly to the collection canister. Conduit 2016 includes a main lumen 2110 used to provide a pathway for exudate between the wound “w₂” and the collection canister. A secondary lumen or vent lumen is provided in conduit 2016 to provide a controlled leak to the wound site. Exudate enters lumen 2110 at area 2114 and air exits lumen 2112 at area 2116. Secondary lumen 2112 is exposed to the ambient environment or to a source of air to provide a controlled leak in the wound port 2000. Although FIG. 27B depicts lumens 2110 and 2112 in a single conduit 2016, lumens 2110 and 2112 can be provided as separate conduits.

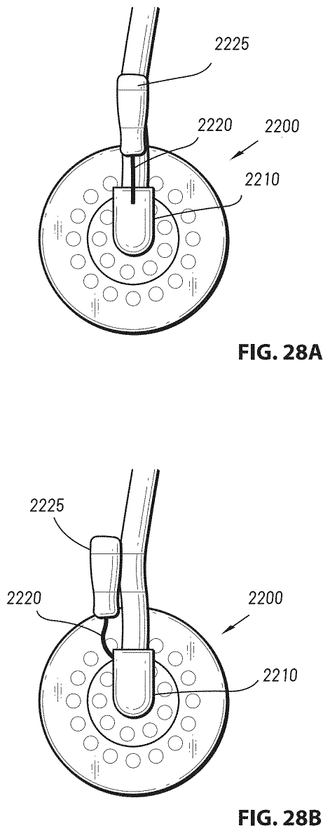

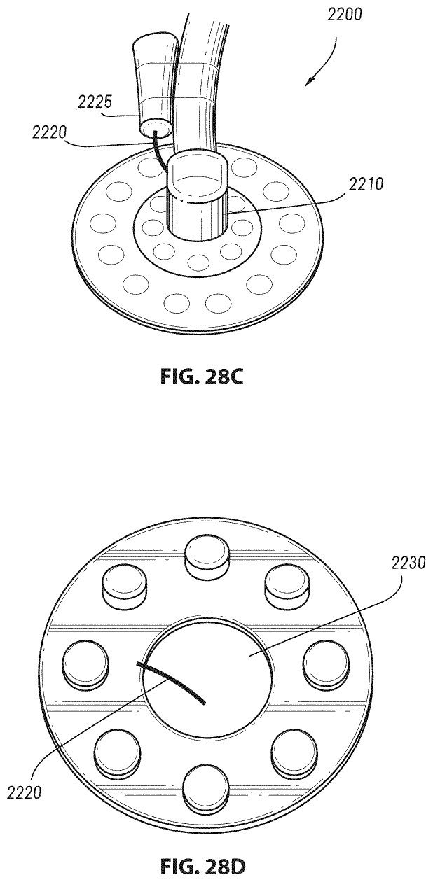

With reference to FIG. 28A, in yet another embodiment, a wound dressing 2200 is shown having a wound port 2210. Wound dressing 2200 and wound port 2210 are similar to wound dressing 1012, 1112, and 1212 and wound port 2210 are similar to wound port 1030, 1130 and described hereinabove. A vent conduit 2220 may be inserted into the top of wound port 2210 to provide a source of filtered air into the wound dressing 2200 through the vent conduit 2220. Vent conduit 2220 may be a stainless steel needle having a lumen extending through the needle. The end of vent conduit 2220 has filter 2225 to filter the air from the ambient atmosphere. The low flow filtered air flowing from the ambient atmosphere through the vent conduit 2220, in combination with the high flow drainage occurring through an exudate conduit, creates a sump action between the wound and a collection canister. This sump action ensures continuous flow through exudate conduit 1036, thereby preventing fluid stagnation and its complications. As discussed above, the difference in size between exudate conduit and vent conduit 2220 results in capillary action that causes fluid to flow only through the larger exudate conduit. FIGS. 28B-28D depict a wound port 2210 similar to the wound port in FIG. 28A. In FIGS. 28B-28D, the vent conduit 2220 is placed on the side of the wound port 2210 rather than the top of the wound port 2210 as shown in FIG. 28A. FIG. 28D depicts the end of vent conduit 2220 being located in the wound port 2210 above an exudate orifice 2230.

Although the foregoing disclosure has been described in some detail by way of illustration and example, for purposes of clarity or understanding, it will be obvious that certain changes and modifications may be practiced within the scope of the appended claims. For example, the individual fluid and vent conduits may be substituted for by a conduit having a dual lumen. To ensure the capillary action, one lumen must be larger than the other; however, the lumens may be coaxial or parallel.

1. A wound dressing comprising:
a backing layer for positioning over a wound to define a reservoir in which a reduced pressure may be maintained over the wound; and
a portal member affixed to the backing layer such that an ambient surface of the portal member may be fluidly isolated from a reservoir surface of the portal member, the backing layer defining a boundary between the ambient surface and the reservoir surface when the backing layer is positioned over a wound, the portal member comprising:
a primary port extending between an opening in the ambient surface and a primary aperture in the reservoir surface, the opening adapted for connection to a suction device and the primary aperture adapted for establishing fluid communication with the reservoir; and
at least one supplemental port establishing fluid communication between the primary port and a supplemental aperture in the reservoir surface, the supplemental aperture distinct and substantially spaced from the primary aperture.

2. The wound dressing according to claim 1, wherein the at least one supplemental port includes a plurality of supplemental ports, each supplemental port extending radially outwardly from the primary port.

3. The wound dressing according to claim 1, wherein the at least one supplemental port includes a plurality of supplemental ports, each supplemental port extending between the central port and a respective supplemental aperture in the reservoir surface, such that the primary and supplemental apertures are distributed substantially over the entire reservoir surface.

4. The wound dressing according to claim 1, wherein the portal member is molded from flexible polymeric or elastomeric material.

5. The wound dressing according to claim 1, wherein the portal member further comprises an attachment flange adapted for connection to the backing layer.

6. The wound dressing according to claim 1, wherein the backing layer comprises a flexible polymeric membrane.

7. The wound dressing according to claim 7, wherein the backing layer comprises an adhesive coating around a periphery thereof, the adhesive coating adapted to affix the backing layer over the wound and to provide a substantially fluid tight seal around the perimeter of the wound.

8. The wound dressing according to claim 8, wherein the adhesive coating is interrupted such that an intermediate portion of the backing layer remains uncoated.

9.-30. (canceled)
